(19) United States
(12) Patent Application Publication
(10) Pub. No.: US 2024/0318257 A1
(43) Pub. Date: Sep. 26, 2024

(54) COLORECTAL CANCER MOLECULAR TYPING AND SURVIVAL RISK FACTOR GENE CLUSTER, DIAGNOSTIC PRODUCT, AND APPLICATION

(71) Applicant: HANGHAI PRECISION MEDICINE CO., LTD.., Shanggai (CN)

(72) Inventors: Tong ZHOU, Shanghai (CN); Zhiyuan HU, Shanghai (CN); Jeff Weiqing CHOU, Shanghai (CN); Linlin MA, Shanghai (CN); Junhuan LU, Shanghai (CN)

(73) Assignee: SHANGHAI PRECISION DIAGNOSTICS CO., LTD., Shanghai (CN)

(21) Appl. No.: 18/269,451

(22) PCT Filed: Dec. 24, 2021
(86) PCT No.: PCT/CN2021/141033

§ 371 Date: Mar. 21, 2024

(30) Foreign Application Priority Data
Dec. 25, 2020 (CN) 202011561310.2

(51) Int. Cl. C12Q 1/6886

(52) U.S. Cl. CPC C12Q 1/6886; C12Q 2600/112; C12Q 2600/158

(57) ABSTRACT

Disclosed are a gene panel for molecular subtyping and assessing the survival risk of colorectal cancer, and use of agents for detecting the expression levels of the genes in the gene panel in the manufacture of a product, the product being used for determining the molecular subtype of colorectal cancer and assessing the survival risk of a patient with colorectal cancer; the product comprises a Next-Generation Sequencing (NGS) detection kit, a fluorescence quantitative PCR detection kit, a gene chip, and a protein chip. Also disclosed is a method for using the detection kit to molecular subtype and assess the survival risk of colorectal cancer.

The application is a National Stage of International Application No. PCT/CN2021/141033 filed Dec. 24, 2021, claiming priority based on Chinese Patent Application No. 202011561310.2, filed on Dec. 25, 2020, entitled “Colorectal cancer molecular typing and survival risk factor gene panel, diagnostic product, and application”, of which the content is incorporated herein in its entirety by reference.

### TECHNICAL FIELD

The present disclosure relates to the field of biotechnology and particularly relates to gene panels for molecular subtyping of colorectal cancer and assessing the survival risk of a patient with colorectal cancer, in vitro diagnostic products, and applications thereof.

### BACKGROUND

The clinical stage of colon cancer is closely related to the therapeutic regimen. Stage I and stage IV colon cancers generally have clear treatment, where stage I is mainly surgery and no adjuvant chemotherapy is needed, while stage IV requires a combined therapy based on chemotherapy. However, the treatment of stage II and III colon cancers is relatively complicated and there is no good predictor for the benefit of chemotherapy after surgery in the current clinical or case diagnosis. Even for patients with identical pathological tissue type and clinical stage, their prognosis varies under the same treatment. It is desirable to have novel biological indicators to guide postoperative adjuvant therapy or preoperative neoadjuvant therapy for such group of patients. In recent years, the development of molecular tumor diagnostic product based on gene expression profiling has provided a new direction for the precise treatment of colon cancer.

The NCCN Clinical Practice Guideline in Oncology (2020.v4) proposes three gene expression profiling-based molecular diagnostic products for colon cancer, Oncotype Dx, ColoPrint and ColDx, to predict the risk of distant metastasis and the benefit of adjuvant chemotherapy after surgery for colon cancer. Oncotype Dx predicts the risk of recurrence of stage II and stage III colorectal cancers and the need for and choice of chemotherapy after surgery by determining the expression profile of 12 genes, and it can also assess postoperative survival in stage II rectal cancer (see Reimers, M. S. et al., 2014, Journal of the National Cancer Institute, 106); ColoPrint, which is an 18-gene expression profiling assay, is also useful for stage II colon cancer recurrence risk assessment; and ColDx, which is a microarray-based 643-gene expression profiling assay is useful for stage II colon cancer recurrence risk assessment. The common feature of the three products lies in that the risk assessment index is an independent prognostic indicator, independent of other risk factors, including TNM stage, tumor grade, lymph node metastasis, mismatch repair (MMR) status, perforation, or the like.

In addition to the recurrence risk assessment, colorectal cancer molecular subtyping based on expression profiles can be used to categorize colorectal cancers into different molecular subtypes, further characterizing molecular features of the tumor and possible mechanism of tumorigenesis and thereby providing targeted clinical treatment regimen or direction for targeted drug development. A consortium of six research institutions engaged in molecular subtyping of colorectal cancer based on gene expression has proposed a consensus molecular subtyping method “CMS” by combining their findings (see Guinney J. et al., The consensus molecular subtypes of colorectal cancer[J]. Nature medicine. 2015, 21(11):1350-6). CMS molecular subtypes include CMS1 (microsatellite instability plus immune activation, 14%), characterized by hypermutation, microsatellite instability (MST), and strong immune activation; CMS2 (classic, 37%), characterized by epithelial phenotype, chromosomal instability, and activation of WNT and MYC signaling pathways; CMS3 (metabolic, 13%), characterized by epithelial phenotype with significant metabolic dysregulation; CMS4 (mesothelial, 23%), characterized by TGFβ activation, stromal invasion, and angiogenesis; and the mixed subtype (13%), which may represent an unknown subtype or intra-tumor heterogeneity. However, there is no significant difference in survival data (OS, DFS) among subtypes in the CMS subtyping system, especially among CMS1 to CMS3.

### SUMMARY

In an aspect, provided is a gene panel for determining the molecular subtype of colorectal cancer and/or assessing the survival risk of a patient with colorectal cancer, comprising molecular subtyping and survival risk assessing related genes. In an embodiment, the gene panel further comprises a reference gene(s). The molecular subtypes of colorectal cancer include a CRC1 subtype, a CRC2 subtype, a CRC3 subtype, a CRC4 subtype, a CRC5 subtype and a mixed subtype.

In an aspect, provided is an agent for detecting expression levels of the genes in the gene panel according to the present disclosure. In a preferable embodiment, the agent is an agent for detecting the amount of RNA, particularly mRNA, transcribed from the genes according to the present disclosure, or an agent for detecting the amount of cDNA complementary to the mRNA. In a specific embodiment, the agent is a primer(s), a probe(s) or a combination thereof.

In another aspect, provided is a product for determining the molecular subtype of colorectal cancer and/or assessing the survival risk of colorectal cancer, comprising the agent according to the present disclosure. Provided is also use of the gene panel or agent according to the present disclosure in the manufacture of a product. The product is useful for determining the molecular subtype of colorectal cancer and/or assessing the survival risk of a patient with colorectal cancer. In an embodiment, the product is a Next-Generation Sequencing kit, a Real-time fluorescence quantitative PCR detection kit, a gene chip, a protein microarray, an ELISA diagnostic kit or an Immunohistochemistry (IHC) kit. In a preferable embodiment, the product is a Next-Generation Sequencing kit or a Real-time fluorescence quantitative PCR detection kit.

In an aspect, provided is a method for determining the molecular subtype of colorectal cancer and/or assessing the survival risk of a subject, comprising (1) providing a sample of the subject; (2) determining expression levels of the genes in the gene panel according to the present disclosure in the sample; and (3) determining the molecular subtype of colorectal cancer and/or survival risk of the subject.

### BRIEF DESCRIPTION OF THE DRAWINGS

FIG. 1 shows an expression heatmap of the colorectal cancer molecular subtyping and survival risk assessing related genes (proliferation-related genes, extracellular matrix-related genes, intracellular matrix-related genes, immune-related genes, and immunoglobulin-related genes) in CRC1 subtype, CRC2 subtype, CRC3 subtype, CRC4 subtype, CRC5 and Mixed subtype.

FIG. 2 shows the results of a survival analysis of 1091 colorectal cancer cases (categorized as CRC1 subtype, CRC2 subtype, CRC3 subtype, CRC4 subtype, CRC5 subtype and Mixed subtype) using the Kaplan-Meier method, indicating that the survival risk for each subtype of colorectal cancer varies, where the CRC2 subtype shows a good 10-year metastasis-free survival rate, the CRC1 subtype and CRC5 subtype show relatively poor 10-year metastasis-free survival rates, the CRC3 subtype and CRC4 subtype show medium prognosis.

FIG. 3 shows the results of a survival analysis of 1091 colorectal cancer cases (categorized as two groups with strong immunoglobulin index and weak immunoglobulin index) using the Kaplan-Meier method, indicating that the immunoglobulin index can be used to indicate the prognosis of colorectal cancer. Based on the immunoglobulin index, colorectal cancer cases can be categorized as two groups: strong immunoglobulin index and weak immunoglobulin index, with the strong immunoglobulin index group having a higher 10-year metastasis-free survival rate.

FIG. 4 shows the results of a survival analysis of 1091 colorectal cancer cases (categorized as two groups: low and high risk) using a risk assessment model based on Cox model, indicating that the colorectal cancer recurrence risk index can be used to indicate the survival risk. The low-risk group (recurrence risk index 0-65) shows a higher metastasis-free survival rate, and the high-risk group (recurrence risk index 66-100) shows a lower 10-year metastasis-free survival rate.

FIG. 5A shows the results of a survival analysis using the Kaplan-Meier method for stage III colon cancer cases whose survival risks are assessed as high risk (173 cases) (categorized as two groups: with and without chemotherapy), indicating that for stage III colon cancer cases whose survival risks are assessed as high risk, the 10-year metastasis-free survival rate is higher in the group of cases with chemotherapy than that in the group of cases without chemotherapy.

FIG. 5B shows the results of a survival analysis using the Kaplan-Meier method for stage III colon cancer cases whose survival risks are assessed as low risk (108 cases) (categorized as two groups: with and without chemotherapy), indicating that for stage III colon cancer cases whose survival risks are assessed as low risk, there is no significant difference in 10-year distant metastasis-free survival rate between the groups of cases with and without chemotherapy.

### DETAILED DESCRIPTION

### General Definition and Terms

The present disclosure will be described in details below, and it should be noted that the description is provided for the purposed of illustration rather than limitation.

Unless otherwise stated, the technical and scientific terms used herein have the same meaning as commonly understood by a person skilled in the art. If there is a contradiction, the definition provided in this application shall prevail. The experimental methods that are not specified herein, can usually, for example follow the conventional conditions those described in Sambrook et al., Molecular Cloning: A Laboratory Manual, 4^{th} ed, Cold Spring Harbor, N.Y., 2012, or according to the those recommended by the manufacturer.

When a certain amount, concentration, or other value or parameter is set forth in the form of a range, a preferred range, or a preferred upper limit or a preferred lower limit, it should be understood that it is equivalent to specifically revealing any range formed by combining any upper limit or preferred value with any lower limit or preferred value, regardless of whether the said range is explicitly recited. Unless otherwise stated, the numerical ranges listed herein are intended to include the endpoints of the range and all integers and fractions (decimals) within the range.

When used with a numerical variable, the term “approximate” or “about” usually refers to the value of the variable and all the values of the variable within the experimental error (for example, within an average 95% confidence interval) or within +10% of the specified value, or a wider range.

The term “optional” or “optionally” means a subsequently described event or circumstance may or may not occur and that the description includes instances when the event or circumstance occurs and instances in which it does not.

The expression “comprise” or its synonyms “contain”, “include”, “have” or the like are meant to be inclusive, which does not exclude other unlisted elements, steps or ingredients. The expression “consist of” excludes any unlisted elements, steps or ingredients. The expression “substantially consist of” refers to specified elements, steps or ingredients within a given range, together with optional elements, steps or ingredients which do not substantively affect the basic and novel feature of the claimed subject matter. It should be understood that the expression “comprise” encompasses the expressions “substantially consist of” and “consist of”.

The expression “at least one” or “one or more” refers to 1, 2, 3, 4, 5, 6, 7, 8, 9 or more.

The detection of gene expression level herein can be achieved, for example, by detecting a target nucleic acid (e.g., an RNA transcript), or, for example, by detecting the amount of a target polypeptide (e.g., an encoded protein), e.g., using proteomics method to detect protein expression level. The amount of a target polypeptide, such as the amount of a polypeptide, a protein or a protein fragment encoded by a target gene, can be normalized against the amount of the total protein in the sample or the amount of the polypeptide encoded by the reference gene. The amount of a target nucleic acid, such as the DNA of a target gene, its RNA transcript or the amount of cDNA complementary to the RNA transcript, can be normalized against the amount of the total DNA, total RNA or total cDNA in the sample, or the amount of the DNAs, RNA transcripts of a set of reference genes or cDNAs complementary to the RNA transcripts.

The term “polypeptide” herein refers to a compound composed of amino acids connected by peptide bonds, including a full-length polypeptide or an amino acid fragment thereof “Polypeptide” and “protein” can be used interchangeably herein.

The term “nucleotide” comprises deoxyribonucleotide and ribonucleotide. The term “nucleic acid” refers to a polymer composed of two or more nucleotides, encompassing deoxyribonucleic acid (DNA), ribonucleic acid (RNA) and nucleic acid analog.

The term “RNA transcript” refers to total RNA, that is, coding or non-coding RNA, including RNA directly derived from a tissue or a peripheral blood sample and RNA indirectly derived from a tissue or a blood sample after cell lysis. Total RNA includes tRNA, mRNA and rRNA, where mRNA includes that transcribed from a target gene and that from other non-target gene. The term “mRNA” can include precursor mRNA and mature mRNA, either the full-length mRNA or its fragment. The RNA herein that can be used for detection is preferably mRNA, and more preferably mature mRNA. The term “cDNA” refers to DNA with a base sequence complementary to RNA. Those skilled in the art can apply methods known in the art to obtain the RNA transcript and/or cDNA complementary to its RNA transcript from the DNA of a gene, for example, by a chemical synthesis method or a molecular cloning method.

A target nucleic acid (e.g., RNA transcript) herein can be detected and quantified, for example, by hybridization, amplification or sequencing. For example, the RNA transcript is hybridized with a probe(s) or a primer(s) to form a complex, and the amount of the target nucleic acid is obtained by detecting the amount of the complex. The term “hybridization” refers to the process of combining two nucleic acid fragments via stable and specific hydrogen bonds to form a double helix complex under appropriate conditions.

The term “amplification primer” or “primer” refers to a nucleic acid fragment containing 5-100 nucleotides, preferably, 15-30 nucleotides capable of initiating an enzymatic reaction (e.g., an enzymatic amplification reaction).

The term “(hybridization) probe” refers to a nucleic acid sequence (can be a DNA or an RNA) that includes at least 5 nucleotides, for example, 5-100 nucleotides and can hybridize to a target nucleic acid (e.g., the RNA transcript of a target gene or amplified product of the RNA transcript, or cDNA complementary to the RNA transcript) to form a complex under specific conditions. A hybridization probe can also include a label for detection. The term “TaqMan probe” is a probe based on TaqMan technology. Its 5′-end carries a fluorescent group, such as FAM, TET, HEX, NED, VIC or Cy5, etc., and its 3′-end carries a fluorescence quenching group (e.g., TAMRA and BHQ group) or non-fluorescence quenching group (TaqMan MGB probe). It has a nucleotide sequence that can hybridize to a target nucleic acid and can report the amount of nucleic acid forming a complex with it when applied to Real-time fluorescence quantitative PCR (RT-PCR).

The term “reference gene” or “internal reference gene” herein refers to a gene that can be used as a reference to correct and normalize the expression level of a target gene. The reference gene inclusion criteria that can be considered are: (1) the expression in tissues is stable, and the expression level is not affected by pathological conditions or drug treatments or less affected; (2) the expression level should not be too high, to avoid a high proportion of the data acquired from the expression data (such as, those obtained through Next-Generation Sequencing), which will affect the accuracy of data detection and interpretation of other genes.

Therefore, an agent that can be used to detect the expression level of the reference gene according to the present disclosure is also encompassed within the protection scope of the present disclosure. Reference gene that can be used in the present disclosure includes but are not limited to “house-keeping gene”. “Reference gene”, “internal reference gene” and “house-keeping gene” can be used interchangeably.

The term “house-keeping gene” refers to a type of genes whose products are necessary to maintain the basic life activities of cells and are continuously expressed in most or almost all tissues at various stages of individual growth, and the expression levels are less affected by environmental factors.

As used herein, the term “colorectal cancer”, also known as rectal cancer or bowel cancer, is a cancer that originates from the colon or rectum. Due to abnormal growth of the cells, it may invade or metastasize to other parts of the body.

As used herein, the term “colorectal cancer molecular subtyping” refers to a method for categorizing colorectal cancer based on the gene expression profile of colorectal cancer tumor tissue.

As used herein, the term “prognosis” refers to the prediction of the course and progression of colorectal cancer, including but not limited to the prediction of survival risk of colorectal cancer. Colorectal cancer with a lower risk of survival has a better prognosis, and vice versa.

As used herein, “survival risk assessment” refers to assessment of the likelihood of disease progression or death of a patient with colorectal cancer due to colorectal cancer and its related causes during a specified period starting from random. The “disease progression” herein includes but is not limited to increase, recurrence and metastasis of tumor cells. The terms “recurrence risk” and “survival risk” herein can be used interchangeably. The terms “recurrence risk” and “survival risk” can be used interchangeably. Risk of Recurrence score (also called recurrence risk index) is calculated herein to carry out survival risk assessment.

### Gene Panels According to the Present Disclosure

In a general aspect, provided is a gene panel, comprising colorectal cancer molecular subtyping and survival risk assessing related genes.

The colorectal cancer molecular subtyping and survival risk assessing related genes according to the present disclosure may comprise: (1) 21 proliferation-related genes, (2) 17 extracellular matrix-related genes, (3) 16 intracellular matrix-related genes, (4) 13 immune-related genes and (5) 9 immunoglobulin-related genes.
(1) Proliferation-related genes: CCNB2, MKI67, RRM1, SPAG5, TOP2A, CKS1B, DNMT1, DTYMK, EZH2, FOXM1, MAD2L1, MCM2, MCM3, MCM6, PCLAF, PLK1, PSRC1, RFC5, SMC4, TMPO and UBE2S;
(2) Extracellular matrix-related genes: AEBP1, COL6A3, HTRA1, MMP2, TIMP3, CLIC4, DPYSL3, EFEMP1, GJA1, LGALS1, LUM, MSN, PALLD, SERPING1, TIMP1, TNC and VIM;
(3) Intracellular matrix-related genes: ADNP, MAPRE1, TMEM189-UBE2V1, CSE1L, EIF2S2, EIF6, NCOA6, PPP1R3D, PRPF6, PSMA7, RALY, RBM39, RNF114, RPS21, TOMM34 and ZMYND8;
(4) Immune-related genes: CCL5, CD2, CXCL13, GZMA, MNDA, BCL2A1, CCL3, CSF2RB, LCP2, PLA2G7, RASGRP1, RHOH and TLR2;
(5) Immunoglobulin-related genes: CD79A, IGKV1-17, IGKV2-28, CD27, IGHM, IGKV4-1, JCHAIN, POU2AF1 and TNFRSF17.

In a specific aspect, provided is a gene panel, comprising colorectal cancer molecular subtyping and survival risk assessing related genes, as described above, (1) one or more of the 21 proliferation-related genes, (2) one or more of the 17 extracellular matrix-related genes, (3) one or more of the 16 intracellular matrix-related genes, (4) one or more of the 13 immune-related genes, and (5) one or more of the 9 immunoglobulin-related genes.

In an embodiment, the gene panel comprises 76 colorectal cancer molecular subtyping and survival risk assessing related genes (see, Table 1), comprising, the 21 proliferation-related genes, 17 extracellular matrix-related genes, 16 intracellular matrix-related genes, 13 immune-related genes, and 9 immunoglobulin-related genes as described above.

In another embodiment, the gene panel comprises 21 colorectal cancer molecular subtyping and survival risk assessing related genes (see, Table 2), comprising 5 proliferation-related genes (CCNB2, MKI67, RRM1, SPAG5 and TOP2A), 5 extracellular matrix-related genes (AEBP1, COL6A3, HTRA1, MMP2 and TIMP3), 3 intracellular matrix-related genes (ADNP, MAPRE1 and TMEM189-UBE2V1), 5 immune-related genes (CCL5, CD2, CXCL13, GZMA and MNDA), and 3 immunoglobulin-related genes (CD79A, IGKV1-17 and IGKV2-28).

In a preferable embodiment, the gene panel may further comprise a reference gene(s).

Preferably, the reference gene(s) is a house-keeping gene(s). House-keeping gene(s) which may be used according to the present disclosure comprises but is not limited to one or more of the following: GAPDH, GUSB, TFRC, MRPL19, PSMC4 and SF3A1. In an embodiment, the gene panel according to the present disclosure may comprise at least one (e.g., 1, 2, 3, 4, 5, 6, 7 or 8), preferably at least 3, most preferably 6 reference genes of the following: GAPDH, GUSB, TFRC, MRPL19, PSMC4 and SF3A1. In a specific embodiment, the reference gene(s) comprises GAPDH, GUSB, TFRC, MRPL19, PSMC4 and SF3A1. In another specific embodiment, the reference gene(s) comprises GAPDH, GUSB and TFRC.

In a preferable embodiment, the gene panel according to the present disclosure comprises the 76 molecular subtyping and survival risk assessing related genes as described above, and reference gene(s). In a specific embodiment, the reference gene(s) comprises GAPDH, GUSB, TFRC, MRPL19, PSMC4 and SF3A1, where the gene panel is as shown in Table 1.

In another preferable embodiment, the gene panel according to the present disclosure comprises the 21 molecular subtyping and survival risk assessing related genes as described above, and reference gene(s). In an embodiment, the reference gene(s) comprises 3 of GAPDH, GUSB, MRPL19, PSMC4, SF3A1 and TFRC. In a specific embodiment, the reference gene(s) comprises GAPDH, GUSB and TFRC, where the gene panel is as shown in Table 2.

TABLE 1

| No. | Function | Gene Name |
|---|---|---|
| 1 | proliferation-related gene | CCNB2 |
| 2 | proliferation-related gene | CKS1B |
| 3 | proliferation-related gene | DNMT1 |
| 4 | proliferation-related gene | DTYMK |
| 5 | proliferation-related gene | EZH2 |
| 6 | proliferation-related gene | FOXM1 |
| 7 | proliferation-related gene | MAD2L1 |
| 8 | proliferation-related gene | MCM2 |
| 9 | proliferation-related gene | MCM3 |
| 10 | proliferation-related gene | MCM6 |
| 11 | proliferation-related gene | MKI67 |
| 12 | proliferation-related gene | PCLAF |
| 13 | proliferation-related gene | PLK1 |
| 14 | proliferation-related gene | PSRC1 |
| 15 | proliferation-related gene | RFC5 |
| 16 | proliferation-related gene | RRM1 |
| 17 | proliferation-related gene | SMC4 |
| 18 | proliferation-related gene | SPAG5 |
| 19 | proliferation-related gene | TMPO |
| 20 | proliferation-related gene | TOP2A |
| 21 | proliferation-related gene | UBE2S |
| 22 | extracellular matrix-related gene | AEBP1 |
| 23 | extracellular matrix-related gene | CLIC4 |
| 24 | extracellular matrix-related gene | COL6A3 |
| 25 | extracellular matrix-related gene | DPYSL3 |
| 26 | extracellular matrix-related gene | EFEMP1 |
| 27 | extracellular matrix-related gene | GJA1 |
| 28 | extracellular matrix-related gene | HTRA1 |
| 29 | extracellular matrix-related gene | LGALS1 |
| 30 | extracellular matrix-related gene | LUM |
| 31 | extracellular matrix-related gene | MMP2 |
| 32 | extracellular matrix-related gene | MSN |
| 33 | extracellular matrix-related gene | PALLD |
| 34 | extracellular matrix-related gene | SERPING1 |
| 35 | extracellular matrix-related gene | TIMP1 |
| 36 | extracellular matrix-related gene | TIMP3 |
| 37 | extracellular matrix-related gene | TNC |
| 38 | extracellular matrix-related gene | VIM |
| 39 | intracellular matrix-related gene | ADNP |
| 40 | intracellular matrix-related gene | CSE1L |
| 41 | intracellular matrix-related gene | EIF2S2 |
| 42 | intracellular matrix-related gene | EIF6 |
| 43 | intracellular matrix-related gene | MAPRE1 |
| 44 | intracellular matrix-related gene | NCOA6 |
| 45 | intracellular matrix-related gene | PPP1R3D |
| 46 | intracellular matrix-related gene | PRPF6 |
| 47 | intracellular matrix-related gene | PSMA7 |
| 48 | intracellular matrix-related gene | RALY |
| 49 | intracellular matrix-related gene | RBM39 |
| 50 | intracellular matrix-related gene | RNF114 |
| 51 | intracellular matrix-related gene | RPS21 |
| 52 | intracellular matrix-related gene | TMEM189-UBE2V1 |
| 53 | intracellular matrix-related gene | TOMM34 |
| 54 | intracellular matrix-related gene | ZMYND8 |
| 55 | immune-related gene | BCL2A1 |
| 56 | immune-related gene | CCL3 |
| 57 | immune-related gene | CCL5 |
| 58 | immune-related gene | CD2 |
| 59 | immune-related gene | CSF2RB |
| 60 | immune-related gene | CXCL13 |
| 61 | immune-related gene | GZMA |
| 62 | immune-related gene | LCP2 |
| 63 | immune-related gene | MNDA |
| 64 | immune-related gene | PLA2G7 |
| 65 | immune-related gene | RASGRP1 |
| 66 | immune-related gene | RHOH |
| 67 | immune-related gene | TLR2 |
| 68 | immunoglobulin-related gene | CD27 |
| 69 | immunoglobulin-related gene | CD79A |
| 70 | immunoglobulin-related gene | IGHM |
| 71 | immunoglobulin-related gene | IGKV1-17 |
| 72 | immunoglobulin-related gene | IGKV2-28 |
| 73 | immunoglobulin-related gene | IGKV4-1 |
| 74 | immunoglobulin-related gene | JCHAIN |
| 75 | immunoglobulin-related gene | POU2AF1 |
| 76 | immunoglobulin-related gene | TNFRSF17 |
| 77 | house-keeping gene | GAPDH |
| 78 | house-keeping gene | GUSB |
| 79 | house-keeping gene | MRPL19 |
| 80 | house-keeping gene | PSMC4 |
| 81 | house-keeping gene | SF3A1 |
| 82 | house-keeping gene | TFRC |

TABLE 2

| No. | Function | Gene Name |
|---|---|---|
| 1 | proliferation-related gene | CCNB2 |
| 2 | proliferation-related gene | MKI67 |
| 3 | proliferation-related gene | RRM1 |
| 4 | proliferation-related gene | SPAG5 |
| 5 | proliferation-related gene | TOP2A |
| 6 | extracellular matrix-related gene | AEBP1 |
| 7 | extracellular matrix-related gene | COL6A3 |
| 8 | extracellular matrix-related gene | HTRA1 |
| 9 | extracellular matrix-related gene | MMP2 |
| 10 | extracellular matrix-related gene | TIMP3 |
| 11 | intracellular matrix-related gene | ADNP |
| 12 | intracellular matrix-related gene | MAPRE1 |
| 13 | intracellular matrix-related gene | TMEM189-UBE2V1 |
| 14 | immune-related gene | CCL5 |
| 15 | immune-related gene | CD2 |
| 16 | immune-related gene | CXCL13 |
| 17 | immune-related gene | GZMA |
| 18 | immune-related gene | MNDA |
| 19 | immunoglobulin-related gene | CD79A |
| 20 | immunoglobulin-related gene | IGKV1-17 |
| 21 | immunoglobulin-related gene | IGKV2-28 |
| 22 | house-keeping gene | GAPDH |
| 23 | house-keeping gene | GUSB |
| 24 | house-keeping gene | TFRC |

In a specific embodiment, the gene panel according to the present disclosure may be used to determine the molecular subtype of colorectal cancer and/or assess the survival risk of a patient with colorectal cancer.

The molecular subtype of colorectal cancer may comprise a CRC1 subtype, a CRC2 subtype, a CRC3 subtype, a CRC4 subtype, a CRC5 subtype and a Mixed subtype. The survival risk may comprise a low risk and a high risk.

A person skilled in the art will understand that the gene panel is not limited to the combinations as listed above. According to the contents of the present disclosure, a person skilled in the art can combine the molecular subtyping and survival risk assessing related genes according to the present disclosure with a reference gene(s) to obtain a gene panel comprising a combination of various genes and such gene panels are also within the scope of the present disclosure.

### Diagnosis Products According to the Present Disclosure

In another aspect, provided are an agent for detecting expression levels of the genes in the gene panel according to the present disclosure and use thereof in the manufacture of a detection/diagnostic product. The gene panel is as shown above.

The agent or the detection/diagnostic product may be used to determine the molecular subtype of colorectal cancer and/or assess the survival risk of a patient with colorectal cancer.

Those skilled in the art will understand that the selection of the agent or product can each correspond to the gene in the gene panel according to the present disclosure. As an example, when multiple options are listed, such as the primer(s) of SEQ ID NO. 165-SEQ ID NO. 212 or the probe(s) of SEQ ID NO. 213-SEQ ID NO. 236, it does not mean that the agent or product according to the present disclosure must contain all of these primers or probes but means that the agent or product will contain those primers or probes corresponding to the genes encompassed therein.

In a preferred embodiment, the agent is used to detect the amount of a target nucleic acid (such as DNA, RNA transcript or cDNA complementary to the RNA transcript of a gene in the gene panel according to the present disclosure), and preferably, to detect the amount of RNA transcript, particularly mRNA of a gene in the gene panel according to the present disclosure, or to detect the amount of cDNA complementary to the mRNA. In an embodiment, the agent is an agent for detecting the amount of RNA transcript, particularly mRNA of a target gene (i.e., a gene in the gene panel according to the present disclosure). In another embodiment, the agent is an agent for detecting the amount of cDNA complementary to the mRNA.

In a preferable embodiment, the agent is a probe(s) or a primer(s) or a combination thereof, which can hybridize to a partial sequence of a target nucleic acid (for example, a gene in the gene panel according to the present disclosure, its RNA transcript or cDNA complementary to the RNA transcript) to form a complex. The probe(s) and primer(s) are highly specific to the target nucleic acid. The probe(s) and primer(s) can be artificially synthesized.

In an embodiment, the agent is a primer(s). In an embodiment, the primer(s) has a sequence as shown in SEQ ID NO. 1-SEQ ID NO. 152 or SEQ ID NO. 1-SEQ ID NO. 164 (also see Table 3). In another embodiment, the primer(s) has a sequence as shown in SEQ ID NO. 165-SEQ ID NO. 206 or SEQ ID NO. 165-SEQ ID NO. 212 (also see Table 4).

In a preferable embodiment, the primer(s) is used for Next-Generation Sequencing, preferably used for targeted sequencing. In a specific embodiment, the primer(s) is used for targeted sequencing and has a sequence as shown in SEQ ID NO. 1-SEQ ID NO. 152 or SEQ ID NO. 1-SEQ ID NO. 164 (Table 3).

In another preferable embodiment, the primer(s) is used for quantitative PCR, preferably Real-time fluorescence quantitative PCR (RT-PCR), for example, SYBR Green RT-PCR based on SYBR Green dye and TaqMan RT-PCR based on TaqMan technology. TaqMan RT-PCR comprises, for example, multiplex RT-PCR and singleplex RT-PCR. In an embodiment, the primer(s) is used for SYBR Green RT-PCR, and has a sequence as shown in SEQ ID NO. 165-SEQ ID NO. 206 or SEQ ID NO. 165-SEQ ID NO. 212 (also see Table 4).

In another embodiment, the primer(s) is used for TaqMan RT-PCR, and has a sequence as shown in SEQ ID NO. 165-SEQ ID NO. 206 or SEQ ID NO. 165-SEQ ID NO. 212 (Table 4). In a specific embodiment, the primer(s) is used in singleplex or multiplex RT-PCR and has a sequence as shown in SEQ ID NO. 165-SEQ ID NO. 206 or SEQ ID NO. 165-SEQ ID NO. 212 (Table 4).

In an embodiment, the primer(s) is used in the manufacture of a detection/diagnostic product. The product is a Next-Generation Sequencing kit based on targeted sequencing or a Real-time fluorescence quantitative PCR kit.

In another embodiment, the agent is a probe(s), including but not limited to a probe(s) used in RT-PCR, in situ hybridization (ISH), DNA blotting or RNA blotting, gene chip detections or the like.

In an embodiment, the probe(s) is a probe(s) used in in situ hybridization. The probe(s) used in in situ hybridization comprises, for example, a probe(s) used in dual-color silver-enhanced in situ hybridization (DISH), DNA fluorescent in situ hybridization (DNA-FISH), RNA fluorescence in situ hybridization (RNA-FISH), chromogenic in situ hybridization (CISH) or the like. The probe(s) can have a label. The label can be a fluorescent group (e.g., Alexa Fluordye, FITC, Texas Red, Cy3, Cy5 etc.), biotin, digoxin or the like. In another embodiment, the probe(s) is used in gene chip detection. The probe(s) can have a label. The label can be a fluorescent group. In a specific embodiment, the probe(s) is used for the manufacture of a detection/diagnostic product, and the product is a gene chip.

In a preferable embodiment, the probe(s) is used in RT-PCR. In an embodiment, the probe(s) is used in TaqMan RT-PCR. In an embodiment, the probe(s) is a TaqMan probe. In an embodiment, the probe(s) has a sequence as shown in SEQ ID NO. 213-SEQ ID NO. 233 or SEQ ID NO. 213-SEQ ID NO. 236 (see also Table 4). In a specific embodiment, the probe(s) is a TaqMan probe having a sequence as shown in SEQ ID NO. 213-SEQ ID NO. 233 or SEQ ID NO. 213-SEQ ID NO. 236.

In an embodiment, the probe(s) is used for the manufacture of a detection/diagnostic product. The product is a Real-time fluorescence quantitative PCR detection kit.

In another embodiment, the agent is a combination of a primer(s) and a probe(s). Preferably, the probe(s) is a TaqMan probe. In an embodiment, the combination of primer(s) and probe(s) is used in RT-PCR, for example, singleplex or multiplex RT-PCR. In an embodiment, the primer(s) has a sequence as shown in SEQ ID NO. 165-SEQ ID NO. 206 or SEQ ID NO. 165-SEQ ID NO. 212. In an embodiment, the probe(s) has a sequence as shown in SEQ ID NO. 213-SEQ ID NO. 233 or SEQ ID NO. 213-SEQ ID NO. 236. In a specific embodiment, the primer(s) has a sequence as shown in SEQ ID NO. 165-SEQ ID NO. 206, and the probe(s) is a TaqMan probe(s) having a sequence as shown in SEQ ID NO. 213-SEQ ID NO. 233. In another specific embodiment, the primer(s) has a sequence as shown in SEQ ID NO. 165-SEQ ID NO. 212, and the probe(s) is a TaqMan probe(s) having a sequence as shown in SEQ ID NO. 213-SEQ ID NO. 236 (see also Table 4).

In an embodiment, the primer(s) and probe(s) are used for the manufacture of a diagnostic product. The diagnostic product is a Real-time fluorescence quantitative PCR detection kit, for example, multipleplex or singleplex Real-time fluorescence quantitative PCR detection kit.

In an alternative embodiment, the agent is used to detect the amount of the polypeptide encoded by the target gene (a gene in the gene panel according to the present disclosure). Preferably, the agent is an antibody, an antibody fragment or an affinity protein, which can specifically bind to the polypeptide encoded by the target gene. More preferably, the agent is an antibody or an antibody fragment that can specifically bind to the polypeptide encoded by the target gene. The antibody, antibody fragment or affinity protein can further carry a label for detection, such as an enzyme (e.g., horseradish peroxidase), a radioisotope, a fluorescent label (e.g., Alexa Fluor dye, FITC, Texas Red, Cy3, Cy5, etc.), a chemiluminescent substance (e.g., luminol), biotin, a quantum dot label (Qdot) or the like. Accordingly, in a preferable embodiment, the agent is an antibody or an antibody fragment that can specifically bind to the polypeptide encoded by the target gene, and optionally has a label for detection, and the label is selected from the group consisting of an enzyme, a radioisotope, a fluorescent label, a chemiluminescent substance, biotin, and a quantum dot label. In an embodiment, the agent is used for the manufacture of a detection/diagnostic product. The product is a protein chip (e.g., Protein microarray), an ELISA diagnostic kit or an Immunohistochemistry (IHC) kit.

Therefore, in another aspect, provided is a product, which is used to determine the molecular subtype of colorectal cancer and/or assessing the survival risk of a patient with colorectal cancer. The product comprises the agent according to the present disclosure. The product can be a Next-Generation Sequencing kit based on targeted sequencing, a Real-time fluorescence quantitative PCR kit, a gene chip, a protein chip, an ELISA diagnostic kit or an Immunohistochemistry (IHC) kit or a combination thereof.

In an embodiment, the product is a diagnostic product based on Next-Generation Sequencing (NGS). In a specific embodiment, the product comprises an agent for detecting the expression level of a gene in the gene panel according to the present disclosure. In an embodiment, the gene panel comprises 82 genes, i.e., the 76 molecular subtyping and survival risk assessing related genes as described above, and 6 house-keeping genes (also see Table 1). In an embodiment, the gene panel according to the present disclosure comprises 24 genes, i.e., the 21 molecular subtyping and survival risk assessing related genes as described above and 3 house-keeping genes, where the 3 house-keeping genes comprise 3 of GAPDH, GUSB, TFRC, MRPL19, PSMC4 and SF3A1. In an embodiment, the gene panel according to the present disclosure comprises 24 genes, i.e., the 21 molecular subtyping and survival risk assessing related genes as described above and 3 house-keeping genes (also see Table 2). In a specific embodiment, the diagnostic product based on Next-Generation Sequencing (NGS) comprises a primer(s) having a sequence as shown in SEQ ID NO. 1-SEQ ID NO. 152 or SEQ ID NO. 1-SEQ ID NO. 164 (see also Table 3).

In another embodiment, the diagnostic product is a diagnostic product based on fluorescence quantitative PCR, preferably Real-time fluorescence quantitative PCR (RT-PCR), e.g., SYBR Green RT-PCR and TaqMan RT-PCR. The TaqMan RT-PCR can for example be multiplex RT-PCR and singleplex RT-PCR. In an embodiment, the diagnostic product comprises an agent for detecting the expression levels of the genes in the gene panel according to the present disclosure. In an embodiment, the gene panel comprises 82 genes, i.e., the 76 molecular subtyping and survival risk assessing related genes as described above and 6 house-keeping genes (see also Table 1). In an embodiment, the gene panel comprises 24 genes, i.e., the 21 molecular subtyping and survival risk assessing related genes as described above and 3 house-keeping gene (see also Table 2). In a specific embodiment, the diagnostic product based on fluorescence quantitative PCR comprises a primer(s) having a sequence as shown in SEQ ID NO. 165-SEQ ID NO. 206 or SEQ ID NO. 165-SEQ ID NO. 212. In another specific embodiment, the diagnostic product based on fluorescence quantitative PCR comprises a TaqMan probe(s) having a sequence as shown in SEQ ID NO. 213-SEQ ID NO. 233 or SEQ ID NO. 213-SEQ ID NO. 236. In a preferable embodiment, the diagnostic product based on fluorescence quantitative PCR comprises a primer(s) having a sequence as shown in SEQ ID NO. 165-SEQ ID NO. 206 and a TaqMan probe(s) having a sequence as shown in SEQ ID NO. 213-SEQ ID NO. 233. In a preferable embodiment, the diagnostic product based on fluorescence quantitative PCR comprises a primer(s) having a sequence as shown in SEQ ID NO. 165-SEQ ID NO. 212 and a TaqMan probe(s) having a sequence as shown in SEQ ID NO. 213-SEQ ID NO. 236 (see also Table 4).

In an embodiment, the product is an in vitro diagnostic product. In a specific embodiment, the product is a diagnostic kit.

In an embodiment, the product is useful for determining the molecular subtype of colorectal cancer and/or assessing the survival risk of a patient with colorectal cancer.

In a preferable embodiment, the product further comprises a total RNA extraction reagent, a reverse transcription reagent, a Next-Generation Sequencing reagent and/or a quantitative PCR reagent.

The total RNA extraction reagent can be a conventional total RNA extraction reagent in the art. The examples comprise but are not limited to RNA storm CD201, Qiagen 73504, Invitrogen K156002 and ABI AM1975.

The reverse transcription reagent can be a conventional reverse transcription reagent in the art and preferably comprise dNTP solution and/or RNA reverse transcriptase. Examples of a reverse transcription reagent comprise but are not limited to NEB M0368L, Thermo K1622 and ABI 4366596.

The Next-Generation Sequencing reagent can be a conventional reagent in the art, provided that it can comply with the requirements for the Next-Generation sequencing. The Next-Generation Sequencing reagent can be commercially available and the examples comprise but are not limited to MiSeq® Reagent Kit v3 (150 cycle) (MS-102-3001), and TruSeq® Targeted RNA Index Kit A-96 Indices (384 Samples) (RT-402-1001) from Illumina. The Next-Generation sequencing is conventional in the art, for example target RNA-seq technology. Accordingly, the Next-Generation Sequencing reagent can further comprise Illumina-customized reagents for constructing a targeted RNA-seq library, for example TruSeq® Targeted RNA Custom Panel Kit (96 Samples) (RT-102-1001).

The quantitative PCR reagent can be a conventional reagent in the art, provided that it can comply with the requirements for the quantitative PCR for the obtained sequences. The quantitative PCR reagent can be commercially available. The quantitative PCR technology can be conventional quantitative PCR technology in the art, preferably Real-time fluorescence quantitative PCR technology, for example SYBR Green RT-PCR and Taqman RT-PCR technology. The PCR reagent preferably further comprises reagents that can be used to construct a quantitative PCR library. Preferably, the quantitative PCR reagent can also comprise Real-time fluorescence quantitative PCR reagents, such as those for SYBR Green RT-PCR (such as SYBR Green premix, e.g., SYBR Green PCR Master Mix) and those for Taqman RT-PCR (such as Tagman RT-PCR Master Mix). Those skilled in the art can select a suitable quantitative PCR reagent according to the quantitative PCR technique used. The detection platform for quantitative PCR detection can be AB17500 Real-time fluorescence quantitative PCR instrument or Roche LightCycler® 48011 Real-time fluorescence quantitative PCR instrument or all other PCR instruments that can perform Real-time fluorescent quantitative detection.

In a specific embodiment, the product is a Next-Generation Sequencing kit based on targeted RNA-seq, comprising a primer(s) having a sequence as shown in Table 3 (SEQ ID NO. 1-SEQ ID NO. 152 or SEQ ID NO. 1-SEQ ID NO. 164), and optionally further comprising one or more of the following: total RNA extraction reagent, reverse transcription reagent and Next-Generation Sequencing reagent. Preferably, the Next-Generation Sequencing reagent is an Illumina-customized reagent for constructing a targeted RNA-seq library.

In yet another specific embodiment, the product is a SYBR Green RT-PCR kit, comprising a primer(s) having a sequence as shown in Table 4 (SEQ ID NO. 165-SEQ ID NO. 206 or SEQ ID NO. 165-SEQ ID NO. 212), and optionally further comprising one or more of the following: total RNA extraction reagent, reverse transcription reagent and SYBR Green RT-PCR reagent.

In another specific embodiment, the product is a TaqMan RT-PCR detection kit, comprising a primer(s) (SEQ ID NO. 165-SEQ ID NO. 206 or SEQ ID NO. 165-SEQ ID NO. 212) and a TaqMan probe(s) (SEQ ID NO. 213-SEQ ID NO. 233 or SEQ ID NO. 213-SEQ ID NO. 236) having a sequence as shown in Table 4, and optionally further comprising one or more of the following: total RNA extraction reagent, reverse transcription reagent and TaqMan RT-PCR reagent.

The diagnostic product according to the present disclosure (preferably in the form of a kit) further preferably comprises a device for extracting the testing sample from a subject; for example, a device for extracting tissue or blood from a subject, preferably any blood collection needle capable of taking blood, syringe, etc. The subject can be a mammal, preferably a human, especially a patient suffering from colorectal cancer.

### Methods and Uses According to the Present Disclosure

In another aspect, provided is also a method for determining the molecular subtype of colorectal cancer and/or the survival risk of a subject, comprising
(1) providing a sample of a subject;
(2) determining the expression levels of the genes in the gene panel according to the present disclosure in the sample;
(3) determining the molecular subtype of colorectal cancer and/or recurrence risk of the subject.

The method according to the present disclosure can be used for diagnostic or non-diagnostic purpose.

The subject in the method according to the present disclosure is a mammal, preferably a human, in particular a patient suffering from colorectal cancer.

The sample used in step (1) is not particularly limited, as long as the expression levels of the genes in the gene panel can be obtained therefrom, for example, the total RNA, total protein or the like, preferably total RNA of the subject can be extracted from the sample. The sample is preferably a sample of tissue, blood, plasma, body fluid or a combination thereof, preferably a tissue sample, in particular a paraffin tissue sample. In a preferable embodiment, the sample is a tumor tissue sample or a tissue sample containing tumor cells. In a preferable embodiment, the sample is a tissue with a high content of tumor cells.

Step (2) can be performed by using methods for determining gene expression levels known in the art. Those skilled in the art can select the sample type and sample amount in step (1) as required and select conventional technology in the art to achieve the determination in step (2). Preferably, the expression levels of target genes (such as the molecular subtyping and survival risk assessing related genes according to the present disclosure) are normalized according to the expression level(s) of a reference gene(s). Methods of normalizing expression levels of genes are well known to those skilled in the art.

In an embodiment, step (2) can be performed by detecting the amount of the polypeptide encoded by the target gene (a gene in the gene panel according to the present disclosure). The detection can be done by reagents as described above and technology known in the art, including but not limited to, enzyme-linked immunosorbent assay (ELISA), chemiluminescence immunoassay technology (e.g., immunochemiluminescence assay, chemiluminescence enzyme immunoassay, electrochemiluminescence immunoassay), flow cytometry and immunohistochemistry (IHC).

In a preferable embodiment, step (2) can be performed by detecting the amount of a target nucleic acid. The detection can be done by the above-mentioned reagents and technology known in the art, including but not limited to molecular hybridization technology, quantitative PCR technology or nucleic acid sequencing technology, etc. Molecular hybridization technologies include but are not limited to ISH technology (such as DISH, DNA-FISH, RNA-FISH, CISH technology, etc.), DNA blotting or RNA blotting technology, gene chip technology (such as microarray chip or microfluidic chip technology), etc., preferably, in situ hybridization technology. Quantitative PCR technologies include but are not limited to semi-quantitative PCR and RT-PCR technology, preferably RT-PCR technology, such as SYBR Green RT-PCR technology and TaqMan RT-PCR technology. Nucleic acid sequencing technologies include but are not limited to Sanger sequencing, Next-Generation Sequencing (NGS), 3^{rd}-Generation sequencing, single-cell sequencing technology, etc., preferably Next-Generation Sequencing, more preferably targeted RNA-seq technology. More preferably, the detection is performed with the agent according to the preset disclosure.

In a preferable embodiment, in step (2), the expression levels of the genes in the gene panel according to the present disclosure are determined by Next-Generation Sequencing technology. In an embodiment, the genes in the gene panel are as shown in Table 1 or Table 2. In an embodiment, the gene panel comprises the 76 molecular subtyping and survival risk assessing related genes as described above and 6 house-keeping genes and can also be found in Table 1. In another embodiment, the gene panel comprises the 21 molecular subtyping and survival risk assessing related genes as described above and 3 house-keeping genes and can also be found in Table 2.

In a specific embodiment, step (2) can comprise:
(2a-1) extracting total RNA from the sample;
(2a-2) converting the optionally purified total RNA into cDNA, which is then prepared into a library ready for Next-Generation Sequencing;
(2a-3) sequencing the library obtained in step (2a-2) and optionally normalizing the expression levels of the molecular subtyping and survival risk assessing related genes according to the expression level(s) of the house-keeping gene(s).

The extraction in step (2a-1) can be performed by conventional methods in the art, preferably using a commercially available RNA extraction kit to extract the total RNA from a fresh frozen tissue or paraffin-embedded tissue of the subject. In a more preferable embodiment, RNA storm CD201 or Qiagen 73504 can be used for extraction.

In a preferable embodiment, step (2a-2) can comprise:
(i) reverse transcribing the extracted total RNA to generate the cDNA of the gene of interest;
(ii) preparing the resulting cDNA into a library ready for sequencing.

In a preferable embodiment, in step (2a-2), the primers shown in Table 3 are used to amplify the cDNA to prepare a library ready for sequencing.

Step (2a-3) can be performed by RNA sequencing. The sequencing method can be a RNA-seq sequencing method conventional in the art for determining gene expression level. Next-Generation Sequencing is preferably performed using Illumina NextSeq/MiSeq/MiniSeq/iSeq series sequencers. The primers in the kit are used to amplify the genes in the gene panel according to the present disclosure, and according to the different libraries prepared in step (2a-2), the Next-Generation Sequencing of the obtained gene sequences can be performed. In an embodiment, the primer pairs in Table 3 are used for sequencing of the genes in Table 1. Preferably, the Next-Generation Sequencing is targeted RNA-seq technology, and the Illumina NextSeq/MiSeq/MiniSeq/iSeq sequencer is used for paired-end sequencing or single-end sequencing. Such a process can be automatically performed by the instrument itself.

In step (2), the expression levels of the genes in the gene panel according to the present disclosure can also be determined by fluorescence quantitative PCR method. In another embodiment, the gene panel comprises the 21 molecular subtyping and survival risk assessing related genes as described above and 3 house-keeping genes and can also be found in Table 2.

In a specific embodiment, step (2) can comprise:
(2b-1) extracting total RNA from the sample;
(2b-2) reverse transcribing the total RNA in (2-1) into cDNA;
(2b-3) subjecting the obtained cDNA to Real-time fluorescence quantitative PCR (RT-PCR) detection, and optionally normalizing the expression levels of the molecular subtyping and survival risk assessing related genes according to the expression levels of the house-keeping genes.

The extraction of step (2b-1) can be performed by conventional methods in the art, preferably using a commercially available RNA extraction kit to extract the total RNA from a fresh frozen tissue or paraffin-embedded tissue of the subject. In a more preferable embodiment, RNA storm CD201 or Qiagen 73504 can be used for extraction. The reverse transcription in step (2b-2) can be performed using a commercially available Reverse transcription kit. In a preferable embodiment, the RT-PCR method in step (2b-3) is TaqMan RT-PCR. Preferably, primers and probes can be used to perform RT-PCR detection of the genes shown in Table 2, and the probes are TaqMan probes. Preferably, the sequences of the primers and probes are as shown in Table 4. In an embodiment, singleplex or multiplex RT-PCR assay is performed using the primers and probes as shown in Table 4.

In an alternative embodiment, the RT-PCR method in step (2b-3) is SYBR Green RT-PCR, and primers and commercially available SYBR Green premix can be used to detect the genes shown in Table 2, separately or simultaneously. Preferably, the sequences of the primers are as shown in SEQ ID NO. 165-SEQ ID NO. 212 (see also Table 4).

The above-described RT-PCR detection can be performed using ABI 7500 Real-time fluorescence quantitative PCR instrument (Applied Biosystems) or Roche LightCycler® 48011. After the reaction, the Ct value of each gene is recorded, representing the expression level of each gene.

In an embodiment according to the present disclosure, step (3) can be performed by statistical analysis of the expression levels of the genes in the gene panel according to the present disclosure in the sample obtained in step (2). Optionally, colorectal cancer molecular subtyping and recurrence risk prediction can be performed based on the single sample prediction method SSP (Single Sample Predictor) (see Hu Z, et al., BMC genomics. 2006, 7:96) and the method optimized by Parker et al., (see Parker J S, et al, Journal of clinical oncology: official journal of the American Society of Clinical Oncology. 2009, 27(8):1160-7). The gene expression data obtained in step (2) are analyzed to obtain the subtype of a single sample, and the recurrence risk can be calculated.

In an embodiment, step (3) comprises molecular subtyping of colorectal cancer, which includes determining the molecular subtype of colorectal cancer of a subject according to the expression level of each gene in the sample of the subject obtained in step (2).

The present inventors analyzed gene expression levels of 1091 colorectal cancer cases with clinical information in the Affymetrix GeneChip expression profile database by the EPIG gene expression profile analysis program (see, Zhou T, et al., 2006. Environ Health Perspect 114 (4), 553-559; Chou J W, et al., 2007. BMC Bioinformatics 8, 427) to obtain the expression profiles of the genes according to the present disclosure. Further, according to the expression profiles of the genes, the method of hierarchical clustering is used to compare the similarity among the detected genes and group the genes; the similarity of the expression profiles among the colorectal cancer samples are compared to classify the colorectal cancers, and the colorectal cancers are categorized into a CRC1 subtype, a CRC2 subtype, a CRC3 subtype, a CRC4 subtype, a CRC5 subtype and a Mixed subtype; the gene expression profiles in the colorectal cancer molecular subtypes are used as standard testing data for molecular subtyping and survival risk assessment of the samples.

The molecular subtypes of colorectal cancer can include a CRC1 subtype, a CRC2 subtype, a CRC3 subtype, a CRC4 subtype, a CRC5 subtype and a Mixed subtype:
the CRC1 subtype is mainly characterized in low expression of proliferation-related genes, high expression of extracellular matrix-related genes, low expression of immune-related genes, low expression of intracellular matrix-related genes and low 10-year metastasis-free survival rate;
the CRC2 subtype is mainly characterized in medium expression of proliferation-related genes, low expression of extracellular matrix-related genes, high expression of immune-related genes, low expression of intracellular matrix-related genes and highest 10-year metastasis-free survival rate;
the CRC3 subtype is mainly characterized in high expression of proliferation-related genes, low expression of extracellular matrix-related genes, low expression of immune-related genes, high expression of intracellular matrix-related genes and medium 10-year metastasis-free survival rate;
the CRC4 subtype is mainly characterized in low expression of proliferation-related genes, low expression of extracellular matrix-related genes, high expression of immune-related genes, low expression of intracellular matrix-related genes and medium 10-year metastasis-free survival rate;
the CRC5 subtype is mainly characterized in medium expression of proliferation-related genes, high expression of extracellular matrix-related genes, low expression of immune-related genes, medium expression of intracellular matrix-related genes and low 10-year metastasis-free survival rate;
the Mixed subtype is the colorectal cancer not belonging to the CRC1 subtype, the CRC2 subtype, the CRC3 subtype, the CRC4 subtype and the CRC5 subtype.

In a specific embodiment, step (3) may comprise determining the colorectal cancer molecular subtype of a subject, comprising
(3-1) according to the expression data of the gene panel according to the present disclosure in a statistically significant number of colorectal cancer samples (training set), establishing the expression profiles of the gene panel according to the present disclosure in the CRC1 subtype, CRC2 subtype, CRC3 subtype, CRC4 subtype and CRC5 subtype as Standard test data;
(3-2) according to the expression levels of the genes in the gene panel according to the present disclosure in the sample obtained in step (2), using the Pearson correlation analysis method, calculating the correlation coefficient between the expression profile of the gene panel according to the present disclosure in the sample and the gene expression profile in the CRC1 subtype, CRC2 subtype, CRC3 subtype, CRC4 subtype or CRC5 subtype in the Standard test data (i.e., the Pearson correlation coefficient between the sample and the CRC1 subtype, CRC2 subtype, CRC3 subtype, CRC4 subtype or CRC5 subtype tumors);
(3-3) when the correlation coefficient between the gene expression profile in the sample and the gene expression profile of X subtype (X is selected from CRC1 subtype, CRC2 subtype, CRC3 subtype, CRC4 subtype or CRC5 subtype) is the highest and the confidence limit is greater than or equal to 0.8, determining said sample as X subtype; and when the confidence limit is lower than 0.8, determining said sample as Mixed subtype.

In another embodiment, step (3) further comprises determining the survival risk of the subject, comprising:
(3a) calculating the immunoglobulin index of the subject according to the the expression level of the immunoglobulin-related genes;
(3b) determining the MMR index of subject according to the mismatch repair status; and
(3c) calculating the survival risk of the colorectal cancer patient.

In an embodiment, step (3) comprises:
(3a-1) according to the expression data of the immunoglobulin-related genes in the gene panel according to the present disclosure in a statistically significant number of colorectal cancer samples (training set), calculating the weighted average value of the expression levels of the immunoglobulin-related genes in the training set, by combination with the survival data, using the statistical software known in the art (such as x-tile software, SPSS or other analysis software that can be used to calculate the cut-off value, preferably x-tile software) for survival analysis, and obtaining a weighted average value that can distinguish the difference in the survival curves to the greatest extent as the cut-off value;
(3a-2) according to the expression levels of the immunoglobulin-related genes obtained in step (2), calculating the weighted average value of the expression levels of the immunoglobulin-related genes in the sample of the subject, i.e., the immunoglobulin index of the subject, and based on the cut-off value in step (3a-1), determining the immunoglobulin index as strong (the expression levels of the immunoglobulin-related genes obtained in step (2)>the cut-off value) or as weak (the expression levels of the immunoglobulin-related genes obtained in step (2)≤the cut-off value);
(3a-3) assessing the recurrence risk according to the immunoglobulin index obtained in step (3a-2): if the immunoglobulin index of the subject is strong, the immune function of the subject is strong, the recurrence risk is low and the prognosis is good; if the immunoglobulin index of the subject is weak, the immune function of the subject is weak, the recurrence risk is high, and the prognosis is poor.

The immunoglobulin index can be calculated according to the following formula:

$$Immunoglobulin index = \frac{1}{n} * {\sum }_{i = 1}^{n} immunoglobulin - related genes ,$$

wherein n is number of the immunoglobulin-related genes for calculating the immunoglobulin index and is an integer of 1-9.

In an embodiment, n=9, the immunoglobulin-related genes comprise CD79A, IGKV1-17, IGKV2-28, CD27, IGHM, IGKV4-1, JCHAIN, POU2AF1 and TNFRSF17 (see also relevant information in Table 1). In another embodiment, n=3, the immunoglobulin-related genes comprise CD79A, IGKV1-17 and IGKV2-28 (see also Table 2).

After obtaining the data on the expression levels of the genes in the gene panel according to the present disclosure, those skilled in the art can apply technology known in the art to obtain a weighted average value of the expression levels of each group of genes and combine the survival data to obtain a weighted value that can distinguish the difference in survival curves to the greatest extent as the cut-off value.

In an embodiment, step (3b) comprises the following steps:
(3b-1) determining the mismatch repair (MMR) status of the subject sample; and
(3b-2) determining the MMR index of the subject based on the MMR status, wherein the MMR index can be assigned by the following formula:
when the MMR status is proficient mismatch repair (pMMR), MMR index=1;
when the MMR status is deficient mismatch repair (dMMR), MMR index=−1.

As used herein, “mismatch repair (MMR)” refers to the process of correcting nucleotide mismatches caused by DNA replication errors, recombination, and certain types of base modifications. MMR proteins (e.g., MLH1, PMS2, MSH2 and MSH6 etc.) perform the function of recognizing and repairing mismatches. In general, MMR status may include deficient mismatch repair (dMMR) and proficient mismatch repair (pMMR).

As used herein, “microsatellite instability (MSI)” refers to any change in the length of a microsatellite due to the insertion or deletion of a repetitive unit compared to a normal microsatellite (MS). In general, it is believed that MSI is resulted from deficient mismatch repair.

The process for determining MMR status can be performed using methods known in the art and may comprise, for example: by detecting expression of MMR proteins (e.g., using immunohistochemistry) and by detecting microsatellite site instability (e.g., using PCR). In some embodiments, the MMR proteins comprise MLH1, PMS2, MSH2 and MSH6. In some embodiments, the microsatellite sites comprise BAT25, BAT26, D5S346, D2S123 and D17S250. In some embodiments, step (3b-1) is conducted by detecting the expression of MLH1, PMS2, MSH2 and MSH6 using immunohistochemistry and/or detecting BAT25, BAT26, D5S346, D2S123 and D17S250 using PCR.

The process for determining MMR status of a sample can be referred to, for example, the Bethesda guideline criteria (J Natl Cancer Inst. 2004 Feb. 18; 96(4): 261-268.). For example, the expression of MLH1, PMS2, MSH2 and MSH6 in a sample may be detected by immunohistochemistry. When the expression of any of these proteins is completely absent, the MMR status of the sample is determined as deficient MMR (dMMR). When there is no absence of the expression of MMR proteins, the MMR status of the sample is determined as proficient MMR (pMMR). Alternatively, microsatellite sties BAT25, BAT26, D5S346, D2S123 and D17S250 may be detected by PCR and compared with normal MS. If at least two sites (e.g., 2, 3, 4 or 5) (i.e., more than 40%) show instability, the MSI of the sample is determined to be high frequency MSI (MSI-H), and the MMR status is dMMR. If one site shows instability, the MSI of the sample is determined to be low frequency MSI (MSI-L), and the MMR status is pMMR. If no instability is detected, the MSI of the sample is determined to be microsatellite stable (MSS), and the MMR status is pMMR.

In an embodiment, step (3) further comprises (3c) calculating the survival risk of the patient with colorectal cancer, comprising the following steps:
(3c-1) using the Cox model, and taking the occurrence and time of disease progression or death as the observation endpoint, according to the relative risk of impact on survival regarding the Pearson correlation coefficients between the sample and the CRC1 subtype, CRC2 subtype, CRC3 subtype, CRC4 subtype or CRC5 subtype tumors obtained in step (3-2), the immunoglobulin index obtained in step (3a-2) and the MMR index obtained in step (3b-2) to determine the corresponding coefficient, calculating the Risk of Recurrence (ROR) of the subject;
(3c-2) according to the Risk of Recurrence score (also called recurrence risk index) calculated in step (3c-1), determining the survival risk of the subject: low risk (the Risk of Recurrence score is 0-65), and high risk (the Risk of Recurrence score is 66-100).

In a specific embodiment, in step (3c-1), 76 colorectal cancer molecular subtyping and survival risk-related genes (see also Table 1) are used to calculate the Risk of Recurrence score of the subject,

ROR=(0.18*CRC1)+(−0.09*CRC2)+(−0.09*CRC3)+(0.07*CRC4)+(0.27*CRC5)+(−0.15*immunoglobulin index)+(0.32*MMR index); wherein,
“CRC1” represents the Pearson correlation coefficient between the tumor and the CRC1 subtype tumor; “CRC2” represents the Pearson correlation coefficient between the tumor and the CRC2 subtype tumor; “CRC3” represents the Pearson correlation coefficient between the tumor and the CRC3 subtype tumor; “CRC4” represents the Pearson correlation coefficient between the tumor and the CRC4 subtype tumor; “CRC5” represents the Pearson correlation coefficient between the tumor and the CRC5 subtype tumor; “immunoglobulin index” is the immunoglobulin index calculated from the 9 immunoglobulin-related genes in Table 1; “MMR index” is the MMR index determined based on the mismatch repair status, where the MMR index is determined as described above.

In another specific embodiment, in step (3c-1), 21 colorectal cancer molecular subtyping and survival risk-related genes (see also Table 2) are used to calculate the Risk of Recurrence score,

ROR=(0.10*CRC1)+(−0.16*CRC2)+(−0.14*CRC3)+(0.21*CRC4)+(0.10*CRC5)+(−0.24*immunoglobulin index)+(0.27*MMR index); wherein,
“CRC1”, “CRC2”, “CRC3”, “CRC4”, “CRC5” and “MMR index” are as defined above; “immunoglobulin index” is the immunoglobulin index calculated from the 3 immunoglobulin-related genes in Table 2.

Accordingly, provided is also use of the agent for detecting the expression levels of the genes in the gene panel according to the present disclosure for determining the molecular subtype of colorectal cancer and/or assessing the survival risk of a patient with colorectal cancer. Provided is also use of the gene panel according to the present disclosure, or the agent for detecting the expression levels of the genes in the gene panel according to the present disclosure in the manufacture of a product for determining the molecular subtype of colorectal cancer and/or assessing the survival risk of a patient with colorectal cancer. In a preferable embodiment, the product is a detection/diagnostic kit. In an embodiment, the product is an in vitro diagnostic product. The agent is as described above. The product is as described above. According to the method or use according to the present disclosure, colorectal cancer may be categorized into different molecular subtypes, which can include a CRC1 subtype, a CRC2 subtype, a CRC3 subtype, a CRC4 subtype, a CRC5 subtype and a Mixed subtype. According to the method or use according to the present disclosure, the survival risk of a patient with colorectal cancer can be assessed, which may include low risk and high risk.

In another aspect, provided is also a set of immunoglobulin-related genes, comprising: CD79A, IGKV1-17, IGKV2-28, CD27, IGHM, IGKV4-1, JCHAIN, POU2AF1 and TNFRSF17 (see also relevant information in Table 1).

The present disclosure further relates to detecting the expression levels of the immunoglobulin-related genes as described above and calculating the immunoglobulin index; wherein the immunoglobulin index can be used to assess the immune status of a patient with colorectal cancer and guide cellular immunotherapy for colorectal cancer. Accordingly, provided is also use of the immunoglobulin-related genes or an agent for detecting the expression levels of the same in the assessment of survival risk of a patient with colorectal cancer.

Exemplary embodiments according to the present disclosure:

1. A gene panel for determining the molecular subtype of colorectal cancer and/or assessing the survival risk of a patient with colorectal cancer, comprising molecular subtyping and survival risk assessing related genes, wherein the molecular subtyping and survival risk assessing related genes comprise:
(1) one or more of the following proliferation-related genes: CCNB2, MKI67, RRM1, SPAG5, TOP2A, CKS1B, DNMT1, DTYMK, EZH2, FOXM1, MAD2L1, MCM2, MCM3, MCM6, PCLAF, PLK1, PSRC1, RFC5, SMC4, TMPO and UBE2S;
(2) one or more of the following extracellular matrix-related genes: AEBP1, COL6A3, HTRA1, MMP2, TIMP3, CLIC4, DPYSL3, EFEMP1, GJA1, LGALS1, LUM, MSN, PALLD, SERPING1, TIMP1, TNC and VIM;
(3) one or more of the following intracellular matrix-related genes: ADNP, MAPRE1, TMEM189-UBE2V1, CSE1L, EIF2S2, EIF6, NCOA6, PPP1R3D, PRPF6, PSMA7, RALY, RBM39, RNF114, RPS21, TOMM34 and ZMYND8;
(4) one or more of the following immune-related genes: CCL5, CD2, CXCL13, GZMA, MNDA, BCL2A1, CCL3, CSF2RB, LCP2, PLA2G7, RASGRP1, RHOH and TLR2; and
(5) one or more of the following immunoglobulin-related genes: CD79A, IGKV1-17, IGKV2-28, CD27, IGHM, IGKV4-1, JCHAIN, POU2AF1 and TNFRSF17.

2. The gene panel according to item 1, comprising 21 molecular subtyping and survival risk assessing related genes, wherein the molecular subtyping and survival risk assessing related genes comprise:
(1) proliferation-related genes: CCNB2, MKI67, RRM1, SPAG5 and TOP2A;
(2) extracellular matrix-related genes: AEBP1, COL6A3, HTRA1, MMP2 and TIMP3;
(3) intracellular matrix-related genes: ADNP, MAPRE1 and TMEM189-UBE2V1;
(4) immune-related genes: CCL5, CD2, CXCL13, GZMA and MNDA; and
(5) immunoglobulin-related genes: CD79A, IGKV1-17 and IGKV2-28.

3. The gene panel according to item 1, comprising 76 molecular subtyping and survival risk assessing related genes, wherein the molecular subtyping and survival risk assessing related genes comprise:
(1) proliferation-related genes: CCNB2, MKI67, RRM1, SPAG5, TOP2A, CKS1B, DNMT1, DTYMK, EZH2, FOXM1, MAD2L1, MCM2, MCM3, MCM6, PCLAF, PLK1, PSRC1, RFC5, SMC4, TMPO and UBE2S;
(2) extracellular matrix-related genes: AEBP1, COL6A3, HTRA1, MMP2, TIMP3, CLIC4, DPYSL3, EFEMP1, GJA1, LGALS1, LUM, MSN, PALLD, SERPING1, TIMP1, TNC and VIM;
(3) intracellular matrix-related genes: ADNP, MAPRE1, TMEM189-UBE2V1, CSE1L, EIF2S2, EIF6, NCOA6, PPP1R3D, PRPF6, PSMA7, RALY, RBM39, RNF114, RPS21, TOMM34 and ZMYND8;
(4) immune-related genes: CCL5, CD2, CXCL13, GZMA, MNDA, BCL2A1, CCL3, CSF2RB, LCP2, PLA2G7, RASGRP1, RHOH and TLR2; and
(5) immunoglobulin-related genes: CD79A, IGKV1-17, IGKV2-28, CD27, IGHM, IGKV4-1, JCHAIN, POU2AF1 and TNFRSF17.

4. The gene panel according to any one of items 1-3, further comprising a reference gene(s); preferably, the reference gene(s) comprises one of, more preferably 3 of, most preferably 6 of GAPDH, GUSB, TFRC, MRPL19, PSMC4 and SF3A1.

5. The gene panel according to item 2, further comprising a reference gene(s); preferably, the reference gene(s) comprises 3 of GAPDH, GUSB, TFRC, MRPL19, PSMC4 and SF3A1; more preferably the reference gene(s) comprises GAPDH, GUSB and TFRC.

6. The gene panel according to item 3, further comprising a reference gene(s); preferably, the reference gene(s) comprises GAPDH, GUSB, TFRC, MRPL19, PSMC4 and SF3A1.

7. An agent for detecting expression levels of the genes in the gene panel according to any one of items 1-6.

8. The agent according to item 7, being an agent for detecting the amount of RNA, particularly mRNA, transcribed from the genes; or an agent for detecting the amount of the cDNA complementary to the mRNA.

9. The agent according to item 7 or 8, being a primer(s), a probe(s) or a combination thereof.

10. The agent according to item 9, being a primer(s), preferably, the primer(s) has a sequence as shown in SEQ ID NO. 1-SEQ ID NO. 164, or a sequence as shown in SEQ ID NO. 165-SEQ ID NO. 212.

11. The agent according to item 9, being a probe(s), preferably, the probe(s) is a TaqMan probe(s); more preferably, the probe(s) has a sequence as shown in SEQ ID NO. 213-SEQ ID NO. 236; most preferably, the probe(s) is a TaqMan probe(s) having a sequence as shown in SEQ ID NO. 213-SEQ ID NO. 236.

12. The agent according to item 9, being a combination of a primer(s) and a probe(s); preferably, the primer(s) has a sequence as shown in SEQ ID NO. 165-SEQ ID NO. 212, and the probe(s) is a TaqMan probe(s) having a sequence as shown in SEQ ID NO. 213-SEQ ID NO. 236.

13. The agent according to item 7, being an agent for detecting the amount of polypeptides encoded by the genes, preferably the agent is an antibody, an antibody fragment or an affinity protein.

14. A product for determining the molecular subtype of colorectal cancer and/or assessing the survival risk of colorectal cancer, comprising the agent according to any one of items 7-13.

15. Use of the gene panel according to any one of items 1-6, the agent according to any one of items 7-13 or the product according to item 14 for determining the molecular subtype of colorectal cancer and/or assessing the survival risk of a patient with colorectal cancer.

16. Use of the gene panel according to any one of items 1-6 or the agent according to any one of items 7-13 in the manufacture of a product for determining the molecular subtype of colorectal cancer and/or assessing the survival risk of a patient with colorectal cancer.

17. The product according to item 14 or the use according to item 16, wherein the product is in a form of an in vitro diagnosis product, preferably a diagnostic kit.

18. The product according to item 14 or the use according to item 16, wherein the product is a Next-Generation Sequencing kit, a Real-time fluorescence quantitative PCR detection kit, a gene chip, a protein microarray, an ELISA diagnostic kit or an Immunohistochemistry (IHC) kit.

19. The product or the use according to item 18, wherein the product is a Next-Generation Sequencing kit, comprising a primer(s) having a sequence as shown in SEQ ID NO. 1-SEQ ID NO. 164, and optionally comprising one or more of the following: a total RNA extraction reagent, a reverse transcription reagent and a Next-Generation Sequencing reagent.

20. The product or the use according to item 18, wherein the product is a Real-time fluorescence quantitative PCR detection kit, comprising a primer(s) having a sequence as shown in SEQ ID NO. 165-SEQ ID NO. 212.

21. The product or the use according to item 20, wherein the Real-time fluorescence quantitative PCR detection kit further comprises a TaqMan probe, and optionally comprises one or more of the following: a total RNA extraction reagent, a reverse transcription reagent and a reagent for TaqMan RT-PCR.

22. The product or the use according to item 21, wherein the Real-time fluorescence quantitative PCR detection kit comprises a primer(s) having a sequence as shown in SEQ ID NO. 165-SEQ ID NO. 212 and a TaqMan probe(s) having a sequence as shown in SEQ ID NO. 213-SEQ ID NO. 236.

23. The product or the use according to item 20, wherein the Real-time fluorescence quantitative PCR detection kit further comprises one or more of the following: a total RNA extraction reagent, a reverse transcription reagent and a reagent for SYBR Green RT-PCR.

24. The gene panel according to any one of items 1-6, the agent according to any one of items 7-13, the product according to any one of items 14 and 17-23, or the use according to any one of items 15-23, wherein the colorectal cancer comprises a CRC1 subtype, a CRC2 subtype, a CRC3 subtype, a CRC4 subtype, a CRC5 subtype and a Mixed subtype.

### Beneficial Effects

Provided are a gene panel for molecular subtyping and/or survival risk assessment of colorectal cancer, an agent for detecting expression levels of the genes in said gene panel, and a method and product for molecular subtyping and/or survival risk assessment of colorectal cancer.

According to the expression levels of the genes in the gene panel according to the present disclosure in colorectal cancer samples, a molecular subtype system for colorectal cancer can be established to classify colorectal cancer into different subtypes and provide more individualized therapy for patients with colorectal cancer belonging to different subtypes. On the other hand, according to the method and use according to the present disclosure, the recurrence risk of a patient with colorectal cancer can be well predicted and the tumor immune status can be effectively assessed, which has important guiding significance for clinical treatment. By combining the subtype, immunoglobulin index, MMR index and risk score, the prognosis of a patient with colorectal cancer can be determined. Colorectal cancer molecular subtyping and risk assessment of a patient with colorectal cancer can be used to screen for superior population for different therapeutic regimens and provide potential therapeutic method. For a patient with a low recurrence risk, further radiotherapy or chemotherapy may be avoided to reduce the incidence of adverse effects and the financial burden of treatment. For a patient with a high recurrence risk, adjuvant chemotherapy, radiotherapy, or biologic therapy should be given in time to maximize the clinical benefit. For an inoperable patient with an advanced disease, the expression profile-based molecular diagnostic can be used to identify a population that may benefit from a treatment regimen, improve treatment efficiency, and avoid ineffective treatment.

As compared with the existing colorectal cancer molecular subtyping methods, the advantage of the present disclosure lies in that not only the colorectal cancer is subtyped, but also the immunoglobulin index and recurrence risk of a patient with tumor are assessed, and the prognosis of a patient with colorectal cancer and possible benefits from the treatment are comprehensively assessed. Another advantage of the present disclosure lies in that multiple selectable genes or gene combinations are provided as complementary embodiments. When the present disclosure is applied to a patient with cancer, if the detection of the expression levels of one or certain genes is invalid or malfunctioning, due to the patient's pathological condition or other reasons (such as one or certain genes are abnormally expressed), multiple alternatives can be used as supplement, such that the detection results based on the present disclosure are more stable and reliable.

### EXAMPLES

The present disclosure is further described below by Examples, which do not limit the present disclosure to the scope of the Examples. The experimental procedures without specific conditions in the following Examples can be selected according to conventional methods and conditions. The reagents and instruments used in the Examples herein are all commercially available.

### Example 1: Screening of the Related Gene Panel for Colorectal Cancer Subtype Classification and Survival Risk Assessment

Procedure: The expression levels of colorectal cancer genes in 1091 cases with clinical information in the Affymetrix gene chip expression database were analyzed through the gene expression profile analysis program EPIG (see, Zhou, Chou et al, 2006. Environ Health Perspect 114 (4), 553-559; Chou, Zhou et al, 2007.BMC Bioinformatics 8, 427), and the proliferation-related genes, extracellular matrix-related genes, intracellular matrix-related genes, immune-related genes, and immunoglobulin-related genes closely related to colorectal cancer survival risk were screened. Genes with large contribution to subtype classification and survival risk in each group of genes were calculated and selected.

Results: A total of 76 genes and 6 house-keeping genes related to colorectal cancer subtype classification and survival risk were screened, i.e., 82-gene testing combination. See Table 1 for a list of genes.

The 82 genes screened were validated for validity and stability in the data of TCGA database with 419 cases of colorectal cancer. The colorectal cancer can be classified into a CRC1 subtype, a CRC2 subtype, a CRC3 subtype, a CRC4 subtype, a CRC5 subtype and a Mixed subtype:
the CRC1 subtype is mainly characterized in low expression of the proliferation-related genes, high expression of the extracellular matrix-related genes, low expression of the immune-related genes, low expression of the intracellular matrix-related genes and low 10-year metastasis-free survival rate;
the CRC2 subtype is mainly characterized in medium expression of the proliferation-related genes, low expression of the extracellular matrix-related genes, high expression of the immune-related genes, low expression of the intracellular matrix-related genes and highest 10-year metastasis-free survival rate;
the CRC3 subtype is mainly characterized in high expression of the proliferation-related genes, low expression of the extracellular matrix-related genes, low expression of the immune-related genes, high expression of the intracellular matrix-related genes and medium 10-year metastasis-free survival rate;
the CRC4 subtype is mainly characterized in low expression of the proliferation-related genes, low expression of the extracellular matrix-related genes, high expression of the immune-related genes, low expression of the intracellular matrix-related genes and medium 10-year metastasis-free survival rate;
the CRC5 subtype is mainly characterized in medium expression of the proliferation-related genes, high expression of the extracellular matrix-related genes, low expression of the immune-related genes, medium expression of the intracellular matrix-related genes and low 10-year metastasis-free survival rate; and
the Mixed subtype is the colorectal cancer not belonging to the CRC1 subtype, the CRC2 subtype, the CRC3 subtype, the CRC4 subtype and the CRC5 subtype.

### Example 2: Testing Combinations of Genes for Molecular Subtyping and Survival Risk Assessment of Colorectal Cancer

From the 82 genes screened in Example 1, the testing combinations were selected for molecular subtyping and survival risk assessment of colorectal cancer.

### 82-Gene Testing Combination:

Procedure: the 82-gene testing combination was used (see Table 1), wherein the gene panel of 76 colorectal cancer molecular subtyping and survival risk related genes (proliferation-related genes: CCNB2, MKI67, RRM1, SPAG5, TOP2A, CKS1B, DNMT1, DTYMK, EZH2, FOXM1, MAD2L1, MCM2, MCM3, MCM6, PCLAF, PLK1, PSRC1, RFC5, SMC4, TMPO and UBE2S; extracellular matrix-related genes: AEBP1, COL6A3, HTRA1, MMP2, TIMP3, CLIC4, DPYSL3, EFEMP1, GJA1, LGALS1, LUM, MSN, PALLD, SERPING1, TIMP1, TNC and VIM; intracellular matrix-related genes: ADNP, MAPRE1, TMEM189-UBE2V1, CSE1L, EIF2S2, EIF6, NCOA6, PPP1R3D, PRPF6, PSMA7, RALY, RBM39, RNF114, RPS21, TOMM34 and ZMYND8; immune-related genes: CCL5, CD2, CXCL13, GZMA, MNDA, BCL2A1, CCL3, CSF2RB, LCP2, PLA2G7, RASGRP1, RHOH and TLR2; immunoglobulin-related genes: CD79A, IGKV1-17, IGKV2-28, CD27, IGHM, IGKV4-1, JCHAIN, POU2AF1 and TNFRSF17) were used to determine the colorectal cancer molecular subtype and assess the survival risk of a patient with colorectal cancer. Six internal reference genes (comprising GAPDH, GUSB, TFRC, MRPL19, PSMC4 and SF3A1) were used as internal reference to normalize the expression levels of the molecular subtyping and survival risk related genes. The 76 colorectal cancer molecular subtyping and survival risk-related genes in Table 1 were used to calculate the recurrence risk index.

### Results

According to the standard test data obtained in Example 1, via the colorectal cancer molecular subtyping method as described above (see steps (3-1) to (3-3) in the “Methods and uses according to the present disclosure” section), using the expression levels of the 76 colorectal cancer molecular subtyping and survival risk-related genes shown in Table 1 (normalized by the expression levels of GAPDH, GUSB, TFRC, MRPL19, PSMC4 and SF3A1), 1091 colorectal cancer cases were subjected to molecular subtyping, and the colorectal tumors were categorized into CRC1 subtype, CRC2 subtype, CRC3 subtype, CRC4 subtype, CRC5 subtype or Mixed subtype.

By calculating the survival number and time of different subtypes, with the observation of distant metastasis of tumor in colorectal cancer cases within 10 years as observed events, the Kaplan-Meier survival curves can be plotted to obtain the 10-year distant metastasis-free survival rate, indicating the recurrence risk of each subtype. The recurrence risks of the subtypes vary, showing that the recurrence risk of each subtype of colorectal cancer is different.

The CRC1 subtype is mainly characterized in low expression of proliferation-related genes, high expression of extracellular matrix-related genes, low expression of immune-related genes, low expression of intracellular matrix-related genes and low 10-year metastasis-free survival rate;
the CRC2 subtype is mainly characterized in medium expression of proliferation-related genes, low expression of extracellular matrix-related genes, high expression of immune-related genes, low expression of intracellular matrix-related genes and highest 10-year metastasis-free survival rate;
the CRC3 subtype is mainly characterized in high expression of proliferation-related genes, low expression of extracellular matrix-related genes, low expression of immune-related genes, high expression of intracellular matrix-related genes and medium 10-year metastasis-free survival rate;
the CRC4 subtype is mainly characterized in low expression of proliferation-related genes, low expression of extracellular matrix-related genes, high expression of immune-related genes, low expression of intracellular matrix-related genes and medium 10-year metastasis-free survival rate;
the CRC5 subtype is mainly characterized in medium expression of proliferation-related genes, high expression of extracellular matrix-related genes, low expression of immune-related genes, medium expression of intracellular matrix-related genes and low 10-year metastasis-free survival rate; and
the Mixed subtype is the colorectal cancer not belonging to the CRC1 subtype, the CRC2 subtype, the CRC3 subtype, the CRC4 subtype and the CRC5 subtype.

### 2. Immunoglobulin Index

According to the standard test data obtained in Example 1, via the above-mentioned immunoglobulin index calculation method (see steps (3a-1) to (3a-3) in the “Methods and uses according to the present disclosure” section), the expression levels of 9 immunoglobulin-related genes CD79A, IGKV1-17, IGKV2-28, CD27, IGHM, IGKV4-1, JCHAIN, POU2AF1 and TNFRSF17 were used to calculate the immunoglobulin index and each subtype was categorized into two groups according to the immunoglobulin index: strong immunoglobulin index group and weak immunoglobulin index group, and the survival difference between the two groups was observed. The results showed that the immunoglobulin index can indicate the prognosis of colorectal cancer. The 10-year metastasis-free survival rate of the case group with strong immunoglobulin index was high and the prognosis was good.

$$Immunoglobulin index = \frac{1}{n} * {\sum }_{i = 1}^{n} Immunoglobulin - related gene ( n = 9 )$$

### 3. MMR Index

According to the MMR index determination method as described above (see steps (3b-1) to (3b-3) in the “Methods and uses according to the present disclosure” section), the MMR status was determined using immunohistochemistry to detect the expression of the MMR proteins MLH1, PMS2, MSH2 and MSH6 and/or PCR to detect the microsatellite sites BAT25, BAT26, D5S346, D2S123 and D17S250, and the MMR index was determined.

### 4. Recurrence Risk Assessment

The calculation of tumor recurrence risk used the Cox model, taking the occurrence of distant metastasis as the observation endpoint, according to the relative risk of impact on survival regarding the Pearson correlation coefficient between the tumor and each subtype, the immunoglobulin index and MMR index to determine the corresponding coefficient so as to calculate the Risk of Recurrence score. The calculation method is as follows:

Calculation of Risk of Recurrence (ROR): the ROR is in the range of 0-100, wherein 0-65 indicates low risk and 66-100 indicates high risk;

ROR=(0.18*CRC1)+(−0.09*CRC2)+(−0.09*CRC3)+(0.07*CRC4)+(0.27*CRC5)+(−0.15*immunoglobulin index)+(0.32*MMR index); wherein,

“CRC1” represents the Pearson correlation coefficient between the tumor and the CRC1 subtype tumor; “CRC2” represents the Pearson correlation coefficient between the tumor and the CRC2 subtype tumor; “CRC3” represents the Pearson correlation coefficient between the tumor and the CRC3 subtype tumor; “CRC4” represents the Pearson correlation coefficient between the tumor and the CRC4 subtype tumor; “CRC5” represents the Pearson correlation coefficient between the tumor and the CRC5 subtype tumor; “immunoglobulin index” is the immunoglobulin index calculated from the 9 immunoglobulin-related genes in Table 1; “MMR index” is the MMR index determined based on the mismatch repair status, where when the MMR status is pMMR, MMR index=1; and when MMR status is dMMR, MMR index=−1.

According to the calculated Risk of Recurrence score, the tumors were categorized into two groups: low risk (0-65) and high risk (66-100). The results showed that the recurrence risk index could indicate the survival risk of a patient with colorectal cancer: the 10-year distant metastasis-free survival rate was higher in the low-risk group and lower in the high-risk group.

### 24-Gene Testing Combination:

The colorectal cancer molecular subtyping method, immunoglobulin index, MMR index and survival risk score for the 24-gene testing combination were calculated similarly to the 82-gene testing combination. The 24-gene testing combination (see Table 2) comprises: 21 colorectal cancer molecular subtyping and survival risk related genes (proliferation-related genes: CCNB2, MKI67, RRM1, SPAG5 and TOP2A; extracellular matrix-related genes: AEBP1, COL6A3, HTRA1, MMP2 and TIMP3; intracellular matrix-related genes: ADNP, MAPRE1 and TMEM189-UBE2V1; immune-related genes: CCL5, CD2, CXCL13, GZMA and MNDA; immunoglobulin-related genes: CD79A, IGKV1-17 and IGKV2-28) were used to determine the colorectal cancer molecular subtype and assess the survival risk of a patient with colorectal cancer. Three internal reference genes (comprising GAPDH, GUSB and TFRC) were used as internal reference to normalize the expression levels of the molecular subtyping and survival risk related genes. The 21 colorectal cancer molecular subtyping and survival risk-related genes in Table 2 were used to calculate the recurrence risk index.

### Results

### 1. Colorectal Cancer Molecular Subtyping

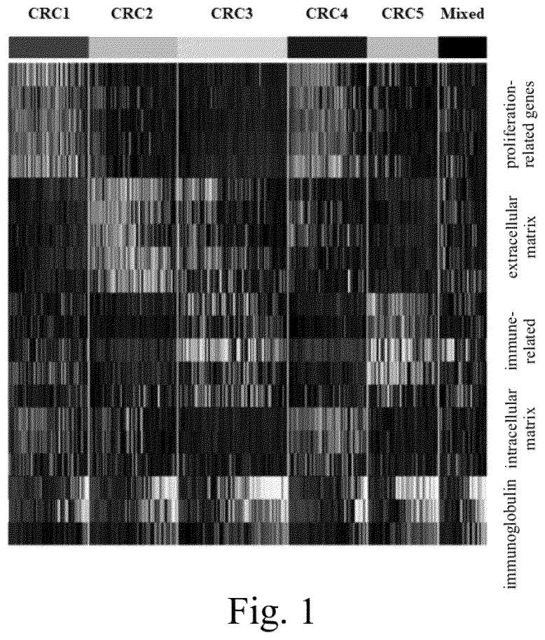

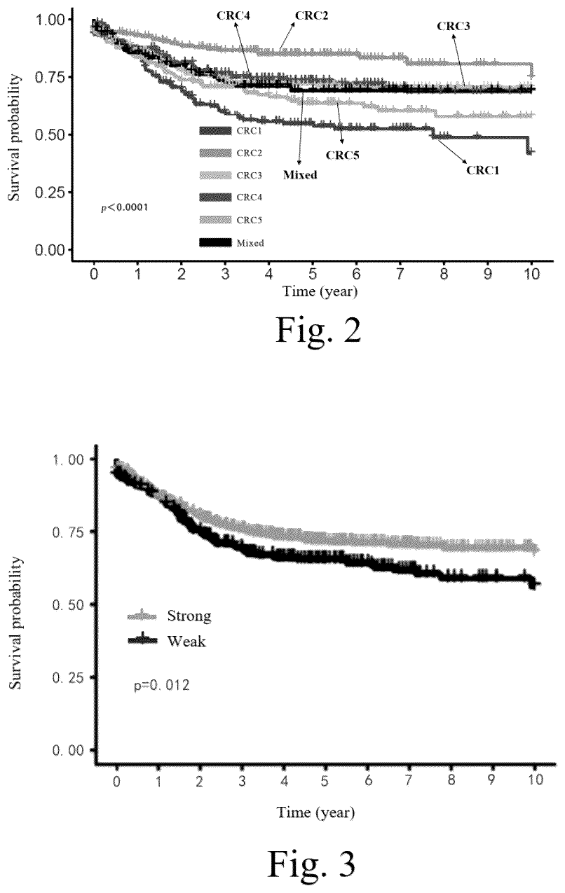

Using the expression levels of the 21 colorectal cancer molecular subtyping and survival risk-related genes (normalized by the expression levels of GAPDH, GUSB and TFRC) shown in Table 2, 1091 colorectal cancer cases were subjected to molecular subtyping, and the colorectal cancer tumors were categorized into CRC1 subtype, CRC2 subtype, CRC3 subtype, CRC4 subtype, CRC5 subtype or Mixed subtype (FIG. 1, FIG. 2). The results were similar to those of the 82-gene testing combination.

### 2. Immunoglobulin Index

The expression levels of 3 immunoglobulin-related genes CD79A, IGKV1-17 and IGKV2-28 were used to calculate the immunoglobulin index and each subtype was categorized into two groups according to the immunoglobulin index: strong immunoglobulin index group and weak immunoglobulin index group and the survival difference between the two groups was observed (FIG. 3). The results were similar to those of the 82-gene testing combination.

$$immunoglobulin index = \frac{1}{n} * {\sum }_{i = 1}^{n} immunoglobulin - related gene ( n = 3 )$$

### 3. MMR Index

According to the MMR index determination method as described above (see steps (3b-1) to (3b-3) in the “Methods and uses according to the present disclosure” section), the MMR status was determined using immunohistochemistry to detect the expression of the MMR proteins MLH1, PMS2, MSH2 and MSH6 and/or PCR to detect the microsatellite sites BAT25, BAT26, D5S346, D2S123 and D17S250, and the MMR index was determined.

### 4. Recurrence Risk Assessment

The calculation of tumor recurrence risk used the Cox model, taking the occurrence of distant metastasis as the observation endpoint, according to the relative risk of impact on survival regarding the subtype of tumor, the immunoglobulin index and MMR index to determine the corresponding coefficient so as to calculate the Risk of Recurrence score. The calculation method is as follows:

ROR=(0.10*CRC1)+(−0.16*CRC2)+(−0.14*CRC3)+(0.21*CRC4)+(0.10*CRC5)+(−0.24*immunoglobulin index)+(0.27*MMR index); wherein,
“CRC1”, “CRC2”, “CRC3”, “CRC4”, “CRC5” and “MMR index” are as defined above; “immunoglobulin index” is the immunoglobulin index calculated from the 3 immunoglobulin-related genes in Table 2.

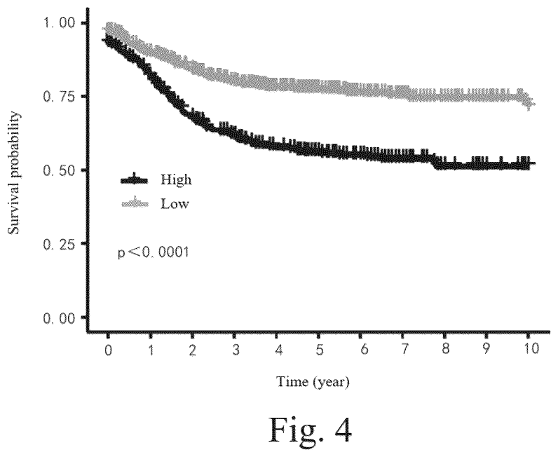

According to the calculated Risk of Recurrence score, the tumors were categorized into two groups: low risk (0-65) and high risk (66-100) (FIG. 4). The results were similar to those of the 82-gene testing combination.

### Example 3: Next-Generation Sequencing Detection Kit for Determining the Molecular Subtype of Colorectal Cancer and Assessing the Survival Risk in a Patient with Colorectal Cancer

According to the 82-gene testing combination in Example 2, a Next-Generation Sequencing detection kit was designed, comprising the primers for specific amplification of the cDNAs of the 82 genes, and the primer sequences are shown in Table 3. The method for determining the molecular subtype of colorectal cancer and assessing the survival risk in a patient with colorectal cancer using the Next-Generation Sequencing detection kit is described below.
Step 1: taking the tumor or paraffin-embedded tissue of the testing subject and using the protocol in the detection kit to obtain the area of the testing subject containing high content of tumor cells as the original material.
Step 2: extracting total RNA from the tissue. RNA storm CD201RNA or Qiagen RNease FFPE kit RNA extraction kit can be used for extraction.
Step 3: preparing the obtained RNA into a library for sequencing. The RNA of the obtained tissue is prepared into a library for Next-Generation Sequencing via targeted RNA-seq technology. The library preparation method comprises the following steps:
(3-1): reverse transcribing the RNA extracted in step (2) into cDNA using ProtoScript® II reverse transcriptase (New England Biolabs, #M0368L).
(3-2): using TruSeq® Targeted RNA Library Construction Kit (#15034457) from Illumina, preparing the obtained cDNA into a library ready for sequencing. The specific steps are as follows: (i) hybridization: adding 4.5 μl of TOP (see Table 3 for the specific compositions), adding 21 μl of OBI after mixing, heating to 70° C. and then slowly cooling to 300° C.; (ii) extension and ligation: adsorbing the product in (i) on a magnetic stand and discarding the supernatant, washing twice with AM1 and UB1 in the kit, discarding the supernatant, adding 36 μl of ELM4, and incubating in a PCR instrument or a metal bath for 37° C. for 45 min; (ii) subjecting the product obtained in (ii) to ligation for the sequencing tag (Index), and then to PCR: adsorbing the product obtained in (ii) on a magnetic stand, discarding the supernatant, add 18 μl of 40-fold diluted HP3, 16 μl of which were taken after absorption with a magnetic stand and added with 17.3 μl of TDP1, 0.3 μl of PMM2, 6.4 μl of Index, and mixing well, where the PCR amplification was performed for 32 cycles; (iv) purifying the DNA using Gnome DNA (QuestGenomics, Nanjing) purification kit to obtain the library.
Step 4: subjecting the obtained DNA library to Next-Generation Sequencing using NextSeq/MiSeq/MiniSeq/iSeq. The Illumina NextSeq/MiSeq/MiniSeq/iSeq sequencer was used to perform paired-end or single-end sequencing. This process was done automatically by the instrument itself (Illumina).
Step 5: Statistical analysis of the results. Performing statistical analysis on the obtained sequencing results; then, subjecting the colorectal cancer of the subject to molecular subtyping by the method described in Example 2, calculating the immunoglobulin index, and Risk of Recurrence score, and predicting the survival risk of the subject.

TABLE 3

| | Gene | | Primer sequence in the | Primer sequence in the |
|---|---|---|---|---|
| No. | Name | Gene ID | upstream region of the gene | downstream region of the gene |
| 1 | CCNB2 | NM_004701 | AATGTGGTGAAAGTAAATGAA | CAAGCAGCAAACTCCTGAAG |
| | | | AACTTAAC (SEQ ID NO. 1) | ATCAG (SEQ ID NO. 2) |
| 2 | CKS1B | NM_001826 | GATGGGTCCATTATATGATCCA | AACCTCACATCTTGCTGTTCC |
| | | | TGAAC (SEQ ID NO. 3) | GG (SEQ ID NO. 4) |
| 3 | DNMT1 | NM_001130823 | TTGGCCAAAGCCCGAGAGAGT | AATAAAGGAGGAGGAAGCTG |
| | | | GCCT (SEQ ID NO. 5) | CTAAG (SEQ ID NO. 6) |
| 4 | DTYMK | NR_033255 | ACCGCGCCGAACTGCTCCGGTT | ATCAACTGAAATCGGCAAAC |
| | | | CCCGGA (SEQ ID NO. 7) | TTCTG (SEQ ID NO. 8) |
| 5 | EZH2 | NM_152998 | CAAGAGGTTCAGACGAGCTGA | AGAGTATGTTTAGTTCCAATC |
| | | | TGAA (SEQ ID NO. 9) | GTCAGAAAA (SEQ ID NO. 10) |
| 6 | FOXM1 | NM_202002 | CGGAGCTACGGCCTAACGGCG | CAATGGAGAGTGAAAACGCA |
| | | | GC (SEQ ID NO. 11) | GATTC (SEQ ID NO. 12) |
| 7 | MAD2L1 | NM_002358 | GCGGGAGCGCCGAAATCGTGG | AACAGCATTTTATATCAGCGT |
| | | | CC (SEQ ID NO. 13) | GGCA (SEQ ID NO. 14) |
| 8 | MCM2 | NM_004526 | TGAGGTCCCTGAGAAGGACTT | ATCCACAACCTCTCTGCATTT |
| | | | GGTG (SEQ ID NO. 15) | TATGACAG (SEQ ID NO. 16) |
| 9 | MCM3 | NM_002388 | GAGATTACCTGGACTTCCTGGA | GGAAGACCAGGGAATTTATC |
| | | | CGA (SEQ ID NO. 17) | AGAGCAAAG (SEQ ID NO. 18) |
| 10 | MCM6 | NM_005915 | AGAAACTGTTCCTGGACTTCTT | TTTCAGAGCAGCGATGGAGA |
| | | | GGA (SEQ ID NO. 19) | AATTAAAT (SEQ ID NO. 20) |
| 11 | MKI67 | NM_002417 | AACCTCTGCTCCCCACCTCAGA | GAGGAAATGTGTTCTTCAGTG |
| | | | GAGTTTT (SEQ ID NO. 21) | CACAG (SEQ ID NO. 22) |
| 12 | PCLAF | NM_014736 | TAAAGCAGACAGTGTTCCAGG | TGGTGGCTGCTCGAGCCCCCA |
| | | | CACT (SEQ ID NO. 23) | GAAA (SEQ ID NO. 24) |
| 13 | PLK1 | NM_005030 | GCAGCGTGCAGATCAACTTCTT | TCACACCAAGCTCATCTTGTG |
| | | | CCA (SEQ ID NO. 25) | CCCA (SEQ ID NO. 26) |
| 14 | PSRC1 | NM_032636 | TGGCTGGACATGGAGGATTTG | ATGTAAGGTTTATTGTGGATG |
| | | | GAGG (SEQ ID NO. 27) | AGACCTTG (SEQ ID NO. 28) |
| 15 | RFC5 | NM_181578 | AATGATCTCATTTCTCATCAGG | TCAATGAAGACCGACTGCCA |
| | | | ACATTC (SEQ ID NO. 29) | CACTT (SEQ ID NO. 30) |
| 16 | RRM1 | NM_001033 | ATGCACTTCTACGGCTGGAAG | GGTTTGAAGACTGGGATGTAT |
| | | | (SEQ ID NO. 31) | TATTTAAG (SEQ ID NO. 32) |
| 17 | SMC4 | NM_005496 | CGCACGGAGAGCCCAGCCACC | AGACTGCAAGTGAGGAACTT |
| | | | GC (SEQ ID NO. 33) | GATAATAG (SEQ ID NO. 34) |
| 18 | SPAG5 | NM_006461 | AGAAAAACTAGATGACATTGT | AGGTGGTGAGGGGATGCAAA |
| | | | TCAGCATA (SEQ ID NO. 35) | GAACT (SEQ ID NO. 36) |
| 19 | TMPO | NM_001032283 | TGAAATACGGAGTGAATCCTG | AGCTATATGAGAAAAAGCTT |
| | | | GTCC (SEQ ID NO. 37) | TTGAAACTGAG (SEQ ID NO. 38) |
| 20 | TOP2A | NM_001067 | AAGAAGACTTGGCTACATTTAT | AAACAAGATGAACAAGTCGG |
| | | | TGAAG (SEQ ID NO. 39) | ACTTCC (SEQ ID NO. 40) |
| 21 | UBE2S | NM_014501 | CCTCACCGACCTCCAGGTCACC | GGACCCCATATGCTGGAGGT |
| | | | ATC (SEQ ID NO. 41) | CTGTT (SEQ ID NO. 42) |
| 22 | AEBP1 | NM_001129 | GGCAAGCCAGGGAAGCGGCCA | \|GCCTCCGGAAAAGACCAAAG |
| | | | GGGA (SEQ ID NO. 43) | ACAAA (SEQ ID NO. 44) |
| 23 | CLIC4 | NM_013943 | AAGAGCCCCTCATCGAGCTCTT | GCAGTGATGGTGAAAGCATA |
| | | | CGT (SEQ ID NO. 45) | GGAAA (SEQ ID NO. 46) |
| 24 | COL6A3 | NM_057164 | AGCCCAGGGACACACGCCTTC | TTTGGTGCTTAGACAAATTCA |
| | | | AGGT (SEQ ID NO. 47) | AAATGAGG (SEQ ID NO. 48) |
| 25 | DPYSL3 | NM_001197294 | TTTATGCTGATATTTACATGGA | TTGGAGACAATCTGATTGTTC |
| | | | AGATGGCT (SEQ ID NO. 49) | CTGG (SEQ ID NO. 50) |
| 26 | EFEMP1 | NM_001039348 | AGGACACCGAAGAAACCATCA | ATGCACTGACGGATATGAGT |
| | | | CGTA (SEQ ID NO. 51) | GGGAT (SEQ ID NO. 52) |
| 27 | GJA1 | NM_000165 | CACTTGGCGTGACTTCACTACT | \|TGGTGCCCAGGCAACATGGG |
| | | | TTT (SEQ ID NO. 53) | TGACT (SEQ ID NO. 54) |
| 28 | HTRA1 | NM_002775 | GTCCTGCAGCGCGGAGCCTGC | GGAAGATCCCAACAGTTTGC |
| | | | GG (SEQ ID NO. 55) | GCCAT (SEQ ID NO. 56) |
| 29 | LGALS1 | NM_002305 | CGAGGCGAGGTGGCTCCTGAC | GCTTCGTGCTGAACCTGGGCA |
| | | | GCTA (SEQ ID NO. 57) | AAGA (SEQ ID NO. 58) |
| 30 | LUM | NM_002345 | AAGAATTAACGAAAGCAGTGT | TTTGCCAAAAATGAGTCTAAG |
| | | | CAAGACAG (SEQ ID NO. 59) | TGCA (SEQ ID NO. 60) |
| 31 | MMP2 | NM_004530 | CTGGATGCCGTCGTGGACCTGC | TCAAGGGTGCCTATTACCTGA |
| | | | AGG (SEQ ID NO. 61) | AGCT (SEQ ID NO. 62) |
| 32 | MSN | NM_002444 | ACTCCGCTGCCTTTGCCGCCAC | CAGTGTGCGTGTGACCACCAT |
| | | | CAT (SEQ ID NO. 63) | GGAT (SEQ ID NO. 64) |
| 33 | PALLD | NM_001166108 | CAAGGAGGACCTCCTGAACAA | AGAAAGAATGGCTCGTCGAC |
| | | | TGGC (SEQ ID NO. 65) | TGCTA (SEQ ID NO. 66) |
| 34 | SERPING1 | NM_001032295 | CTGACCCTGCTGACCCTCCTGC | GAGCCTCCTCAAATCCAAATG |
| | | | TGC (SEQ ID NO. 67) | CTAC (SEQ ID NO. 68) |
| 35 | TIMP1 | NM_003254 | CGCAGATCCAGCGCCCAGAGA | CCTTTGAGCCCCTGGCTTCTG |
| | | | GACA (SEQ ID NO. 69) | GCAT (SEQ ID NO. 70) |
| 36 | TIMP3 | NM_000362 | CCACCCCCAGGACGCCTTCTGC | TCCGGGCCAAGGTGGTGGGG |
| | | | AACTCC (SEQ ID NO. 71) | AAGAA (SEQ ID NO. 72) |
| 37 | TNC | NM_002160 | CTTCCAAGGACCTAGGTCTCTC | AAATAATTCTTTCAAGAAGAT |
| | | | GCC (SEQ ID NO. 73) | CAGGGACA (SEQ ID NO. 74) |
| 38 | VIM | NM_003380 | ACTTCTGATTAAGACGGTTGAA | ATCAACGAAACTTCTCAGCAT |
| | | | ACTAGAG (SEQ ID NO. 75) | CACG (SEQ ID NO. 76) |
| 39 | ADNP | NM_015339 | ACTGTGGGACCCATCACTTACG | TTCTGCTGCAGCGCTTGTCCA |
| | | | AAA (SEQ ID NO. 77) | TTTT (SEQ ID NO. 78) |
| 40 | CSEIL | NM_001316 | GCTGGGGTTCCCTCCTCCGTTT | TCAGCGATGCAAATCTGCAA |
| | | | CTG (SEQ ID NO. 79) | ACACT (SEQ ID NO. 80) |
| 41 | EIF2S2 | NM_003908 | CACTCGAGCCGCAGCCATGTCT | GATTTTTGATCCTACTATGAG |
| | | | GGG (SEQ ID NO. 81) | CAAGAAG (SEQ ID NO. 82) |
| 42 | EIF6 | NM_002212 | CATGCGGGATTCCCTCATTGAC | \|ACCTGAGTCACCTTCCAAGTT |
| | | | AGC (SEQ ID NO. 83) | GTTC (SEQ ID NO. 84) |
| 43 | MAPRE1 | NM_012325 | GGCAGTGGACGCGGTTCTGCC | CGTATACTCAACGTCAGTGAC |
| | | | GAGA (SEQ ID NO. 85) | CAGT (SEQ ID NO. 86) |
| 44 | NCOA6 | NM_014071 | AGAAGATGACCTGGATAAATG | TGTCCTCTTGGCATATGCTTC |
| | | | ATAAAAATTAAG | TGGA (SEQ ID NO. 88) |
| | | | (SEQ ID NO. 87) | |
| 45 | PPP1R3D | NM_006242 | ACCGCAAGAAGCGGAGGACCT | GGAGCAAGGTGGCGAACCAA |
| | | | GGAC (SEQ ID NO. 89) | GGGTA (SEQ ID NO. 90) |
| 46 | PRPF6 | NM_012469 | ACGACGAGGATCTAAATGACA | CTATGCTGGGAGCCTCTTCTC |
| | | | CCAA (SEQ ID NO. 91) | AAGT (SEQ ID NO. 92) |
| 47 | PSMA7 | NM_002792 | TGCTGTCATGAGGCGAGATCA | AGAAATTGAGAAGTATGTTG |
| | | | ATCC (SEQ ID NO. 93) | CTGAAATTG (SEQ ID NO. 94) |
| 48 | RALY | NM_007367 | CGCGGCTTCCTCCAGACCTCTC | GAGGCAGGTGGTGCTGACCC |
| | | | GGC (SEQ ID NO. 95) | TGTAA (SEQ ID NO. 96) |
| 49 | RBM39 | NM_004902 | GAGCACCACAGGCGCCCGAAG | AGAGAAAATGGCAGACGATA |
| | | | GCCG (SEQ ID NO. 97) | TTGATATTG (SEQ ID NO. 98) |
| 50 | RNF114 | NM_018683 | GCACAGAGACTTCTTGCCATG | AAGATCCGGTCCCACGTGGCT |
| | | | GCTG (SEQ ID NO. 99) | ACTT (SEQ ID NO. 100) |
| 51 | RPS21 | NM_001024 | TCTCTCGCGCGCGGTGTGGTGG | AGCCCAGCCTCGAAATGCAG |
| | | | CAG (SEQ ID NO. 101) | AACGA (SEQ ID NO. 102) |
| 52 | TMEM189- | NM_199203 | CCCCACGAGACCTACTTCTGCA | CAATTTCCGACTGTTGGAAGA |
| | UBE2V1 | | TCA (SEQ ID NO. 103) | ACTC (SEQ ID NO. 104) |
| 53 | TOMM34 | NM_006809 | TAATGTGACGTCAGCCGTAGA | TCATGGACTCGCTTGGGCCTG |
| | | | AGGC (SEQ ID NO. 105) | AGTG (SEQ ID NO. 106) |
| 54 | ZMYND8 | NM_183047 | TGACATTACACAGTGTTAACA | CTTGGCTGAAGAGGAAATAA |
| | | | ATGCATCC (SEQ ID NO. 107) | AAACAGAAC (SEQ ID NO. 108) |
| 55 | BCL2A1 | NM_004049 | TTGCCCCGGATGTGGATACCTA | TTTCATATTTTGTTGCGGAGT |
| | | | TAA (SEQ ID NO. 109) | TCATAAT (SEQ ID NO. 110) |
| 56 | CCL3 | NM_002983 | GCTCTCTGCAACCAGTTCTCTG | CTTGCTGCTGACACGCCGACC |
| | | | CAT (SEQ ID NO. 111) | GCCT (SEQ ID NO. 112) |
| 57 | CCL5 | NM_002985 | GCTACTGCCCTCTGCGCTCCTG | CCTCGGACACCACACCCTGCT |
| | | | CATCT (SEQ ID NO. 113) | GCTTT (SEQ ID NO. 114) |
| 58 | CD2 | NM_001767 | CAAGGAATCCAGTGTCGAGCC | TCATCATTGGCATATGTGGAG |
| | | | TGTCA (SEQ ID NO. 115) | GA (SEQ ID NO. 116) |
| 59 | CSF2RB | NM_000395 | AGAAGACTGGTCTCTCCCACC | AGGCCAGGAGGGAGAGGTCC |
| | | | ACAC (SEQ ID NO. 117) | CAAGA (SEQ ID NO. 118) |
| 60 | CXCL13 | NM_006419 | CTGCTGGTCAGCAGCCTCTCTC | TGGAGGTCTATTACACAAGCT |
| | | | CAG (SEQ ID NO. 119) | TGAG (SEQ ID NO. 120) |
| 61 | GZMA | NM_006144 | AAAGACTGGGTGTTGACTGCA | AACAAAAGGTCCCAGGTCAT |
| | | | GCT (SEQ ID NO. 121) | TCTTGG (SEQ ID NO. 122) |
| 62 | LCP2 | NM_005565 | CTGGGACCCCGACAGCCTTGCT | CTGTGAGAAGGCAGTGAAGA |
| | | | GAC (SEQ ID NO. 123) | AGTAC (SEQ ID NO. 124) |
| 63 | MNDA | NM_002432 | CTGAAGACTATTGTGGAAGAA | AAGCTATAACATCAGAAATG |
| | | | GCATCCA (SEQ ID NO. 125) | GTGAATGAA (SEQ ID NO. 126) |
| 64 | PLA2G7 | NM_005084 | AAGCTTCATTAGCATTCTTACA | ATAAAGATTTTGATCAGTGGG |
| | | | AAAGCATT (SEQ ID NO. 127) | ACTG (SEQ ID NO. 128) |
| 65 | RASGRP1 | NM_001128602 | CTGGACGATCTCATTGACAGCT | CCTGTGTCGAAGTAACCAACT |
| | | | GCA (SEQ ID NO. 129) | GTTG (SEQ ID NO. 130) |
| 66 | RHOH | NM_004310 | GAAGCCGGCTACAGGAAATTG | AACTTGCTAATCTCTTTTGTC |
| | | | ACTT (SEQ ID NO. 131) | ACATTCGG (SEQ ID NO. 132) |
| 67 | TLR2 | NM_003264 | ATTGCTCTTTCACTGCTTTCAA | TGAAGCACTGGACAATGCCA |
| | | | CTG (SEQ ID NO. 133) | CATAC (SEQ ID NO. 134) |
| 68 | CD27 | NM_001242 | AAAGCTGTGCTGCCAGATGTG | GAAGGACTGTGACCAGCATA |
| | | | TGAG (SEQ ID NO. 135) | GAAAG (SEQ ID NO. 136) |
| 69 | CD79A | NM_001783 | CCTCTTCCTGCTGTCTGCTGTC | GGTGCCAGGCCCTGTGGATG |
| | | | TAC (SEQ ID NO. 137) | CACAA (SEQ ID NO. 138) |
| 70 | IGHM | X17115.1 | GGGTCACCGAGAGGACCGTGG | AGGGGGAGGTGAGCGCCGAC |
| | | | AC (SEQ ID NO. 139) | GAGGA (SEQ ID NO. 140) |
| 71 | IGKV1-17 | ENSE00002515620 | GAGACAGAGTCACCATCACTT | GCCCCTAAGCGCCTGATCTAT |
| | | | GCCG (SEQ ID NO. 141) | GCTG (SEQ ID NO. 142) |
| 72 | IGKV2-28 | ENSE00002466064 | CAGAGCCTCCTGCATAGTAAT | TGATCTATTTGGGTTCTAATC |
| | | | GGAT (SEQ ID NO. 143) | GGGC (SEQ ID NO. 144) |
| 73 | IGKV4-1 | ENSG00000211598 | CTCCACAGCTCCTGATCTATTT | CACTGAAAATCAGCAGAGTG |
| | | | GGG (SEQ ID NO. 145) | GAGGC (SEQ ID NO. 146) |
| 74 | JCHAIN | NM_144646 | CCTGGCGGTTTTTATTAAGGCT | AAGAAGATGAAAGGATTGTT |
| | | | GTT (SEQ ID NO. 147) | CTTGTTGAC (SEQ ID NO. 148) |
| 75 | POU2AF1 | NM_006235 | TCCTGTCACAGGCCATGCTCTG | ACCCACAGCTCCGGAGCAAG |
| | | | GCA (SEQ ID NO. 149) | CCCCA (SEQ ID NO. 150) |
| 76 | TNFRSF17 | NM_001192 | AATTAACCATTTCGACTCGAGC | ATCTTTTGTCAGAATAGATGA |
| | | | AGT (SEQ ID NO. 151) | TGTGTCAG (SEQ ID NO. 152) |
| 77 | GAPDH | NM_002046 | TCAACGACCACTTTGTCAAGCT | CAGCAACAGGGTGGTGGACC |
| | | | CA (SEQ ID NO. 153) | TCA (SEQ ID NO. 154) |
| 78 | GUSB | NM_000181 | GAGGAGCAGTGGTACCGGCGG | GACATGCCAGTTCCCTCCAGC |
| | | | C (SEQ ID NO. 155) | TTCAAT (SEQ ID NO. 156) |
| 79 | MRPL19 | NM_014763 | CTGTTCTTCCCCTTCGAGGAAT | TCCACGGGGCGGTGCTTGTCC |
| | | | GAA (SEQ ID NO. 157) | ACGA (SEQ ID NO. 158) |
| 80 | PSMC4 | NM_006503 | TCTGGGGCCGGGACACGGACA | CTTCTCCACCAAGATGCCTAT |
| | | | GTGC (SEQ ID NO. 159) | CTCC (SEQ ID NO. 160) |
| 81 | SF3A1 | NM_001005409 | GAATCCTCCTTTGAAGATGCTT | GGCTGTTTGGGCTCCGTGGGC |
| | | | CTT (SEQ ID NO. 161) | ACGG (SEQ ID NO. 162) |
| 82 | TFRC | NM_003234 | GTCATGAAGAAACTCAATGAT | TCCTCTCTCCCTACGTATCTC |
| | | | CGTGTC (SEQ ID NO. 163) | CAAAAG (SEQ ID NO. 164) |

### Example 4: Quantitative PCR Detection Kit for Determining the Molecular Subtype of Colorectal Cancer and Assessing the Survival Risk in a Patient with Colorectal Cancer

According to the 24-gene testing combination in Example 2, a quantitative PCR detection kit was designed, comprising primers for PCR amplification of the 24 genes, and TaqMan probes for quantitative analysis. The sequences of the primers and probes are shown in Table 4. The kit can be used for singleplex or multiplex RT-PCR assay. The method for the molecular subtyping of colorectal cancer and recurrence risk assessment by singleplex RT-PCR assay using the kit is as described below.

Procedures: taking colorectal cancer tumor tissue; extracting RNA from the tumor cells; via the TaqMan RT-PCR technology and with the primers and probes shown in Table 4, detecting gene expression levels respectively. The steps are as follows:
Step 1: taking the tumor or paraffin-embedded tissue of the testing subject and using the protocol in the detection kit to obtain the area of the testing subject containing high contents of tumor cells as the original material.
Step 2: extracting total RNA from the tissue. RNA storm CD201RNA or Qiagen RNease FFPE kit RNA extraction kit can be used for extraction.
Step 3: RT-PCR detection. The RT-PCR detection method is Tagman RT-PCR, and the genes shown in Table 4 are respectively subjected to RT-PCR detection. The steps are as follows:
(3-1): extracting the total RNA of the testing subject;
(3-2): performing reverse transcription on the RNA obtained in (3-1), the specific steps are as follows: taking a total amount of about 2 pg of the sample RNA (for example, taking 11 μl of the sample RNA of about 200 ng/μl), and reverse transcribing it together with 11 μl of the reference RNA (K1622 Reverse Transcription Kit, Thermo) to obtain the sample cDNA and reference cDNA; adding 80 μl of RNase-free water to the sample cDNA for a 5-fold dilution, adding 180 μl of RNase-free water to the reference cDNA for a 10-fold dilution;
(3-3): subjecting the cDNA sample corresponding to each gene obtained in (3-2) to TaqMan RT-PCR to detect the 21 colorectal cancer molecular subtyping and survival risk related genes and 3 reference genes (see Table 2) respectively. The steps are as follows: (i) preparing of a reaction system per well: 2 μl of the obtained cDNA sample (100-400 ng in total) in (3-2), a total of 1.4 μl of the forward and reverse specific primers and the TaqMan fluorescent probes (10 μM) as shown in Table 4, 10 μl of the reaction premix solution, 6.6 μl of DEPC water; (ii) inactivating the reverse transcriptase at 95° C. for 2 min; (iii) amplification and detection: denaturation at 95° C. for 25 sec, annealing at 60° C., elongation and fluorescence detection for 60 sec, 45 cycles, and a holding period at 60° C. for 60 sec; after the amplification reaction, the Ct value of each gene is recorded, representing the expression level of each gene.
Step 4: Statistical analysis of the results. Performing statistical analysis on the obtained sequencing results; then, subjecting the colorectal cancer of the subject to molecular subtyping by the method described in Example 2, calculating the immunoglobulin index and Risk of Recurrence score, and predicting the survival risk.

TABLE 4

| | Gene | | Primer sequence of | Primer sequences of | Probe sequence |
|---|---|---|---|---|---|
| No. | Name | Gene ID | the gene (Forward) | the gene (Reverse) | of the gene |
| 1 | CCNB2 | NM_004701 | AACCAGAGCAGCA | GGTTTGACAGAAGCAG | ACCAAAGTTCCAGTT |
| | | | CAAGTAG | TAGGT | CAACCCACCA |
| | | | (SEQ ID NO. 165) | (SEQ ID NO. 166) | (SEQ ID NO. 213) |
| 2 | MKI67 | NM_002417 | GACCTCAAACTGG | GCTGCCAGATAGAGTC | CGGGAGCAGAGCCAG |
| | | | CTCCTAATC | AGAAAG | TAAACTTCC |
| | | | (SEQ ID NO. 167) | (SEQ ID NO. 168) | (SEQ ID NO. 214) |
| 3 | RRM1 | NM_001033 | TCCACATTGCTGA | CCGCTGGTCTTGTCCTT | AGCAGGGTTTGAAGA |
| | | | GCCTAAC | AAATA | CTGGGATGT |
| | | | (SEQ ID NO. 169) | (SEQ ID NO. 170) | (SEQ ID NO. 215) |
| 4 | SPAG5 | NM_006461 | GCCAGCACCATAG | AGAGAGTCAGGCTCTG | AAAGCTAGGGCTGCT |
| | | | CAGATAA | TAGTT | GACTGAGC |
| | | | (SEQ ID NO. 171) | (SEQ ID NO. 172) | (SEQ ID NO. 216) |
| 5 | TOP2A | NM_001067 | GGAAGATA | ATAA | AAGGCTTGC |
| | | | GACGCTTCGTTATG | GGGCCAGTTGTGATGG | ATGGTTCCCACATCA |
| | | | (SEQ ID NO. 173) | (SEQ ID NO. 174) | (SEQ ID NO. 217) |
| 6 | AEBP1 | NM_001129 | CACCAACGGCTAT | ATTCCAGGTGAGTGGG | TTCATGGGAACGTGG |
| | | | GAGGAAA | TAGA | ACAAGGACA |
| | | | (SEQ ID NO. 175) | (SEQ ID NO. 176) | (SEQ ID NO. 218) |
| 7 | COL6A3 | NM_057166 | CAGGTGAACCTGG | GTCTCCCTTCTGTCCA | ACAACAGGACCCAAA |
| | | | GCTAAAT | ACTATC | GGCATCAGA |
| | | | (SEQ ID NO. 177) | (SEQ ID NO. 178) | (SEQ ID NO. 219) |
| 8 | HTRA1 | NM_002775 | GCTAGTGGGTCTG | TAAGTGGCACCGTTCT | TGGACTGATCGTGAC |
| | | | GGTTTATT | TCAG | AAATGCCCA |
| | | | (SEQ ID NO. 179) | (SEQ ID NO. 180) | (SEQ ID NO. 220) |
| 9 | MMP2 | NM_004530 | AGAGAACCTCAGG | CCTCGAACAGATGCCA | TCTGTCCTGTAGAAA |
| | | | GAGAGTAAG | CAATA | GAGCCCTGAAGA |
| | | | (SEQ ID NO. 181) | (SEQ ID NO. 182) | (SEQ ID NO. 221) |
| 10 | TIMP3 | NM_000362 | TTTGCCCTTCTCCT | TCTTTCACACACCTTG | AGGATCAGTCAAAGG |
| | | | CCAATAC | AGTCTATC | CAGCAAGCA |
| | | | (SEQ ID NO. 183) | (SEQ ID NO. 184) | (SEQ ID NO. 222) |
| 11 | ADNP | NM_015339 | GTCTGCTAATGCCT | TTTGGAACTGGACTGA | TCTCTCAGTCACAGG |
| | | | CTTCTCTC | CCTAAC | CATCCAGAGT |
| | | | (SEQ ID NO. 185) | (SEQ ID NO. 186) | (SEQ ID NO. 223) |
| 12 | MAPRE1 | NM_012325 | GGCTGCGTATTGTC | GTTCTGGATGTACTCG | AATGGAGCCAGGGAA |
| | | | AGTTTATG | TGTTCT | CAGCATGTC |
| | | | (SEQ ID NO. 187) | (SEQ ID NO. 188) | (SEQ ID NO. 224) |
| 13 | TMEM189- | NM_199203 | GCATCAC | TCTTC | CGACTGTT |
| | UBE2V1 | | (SEQ ID NO. 189) | (SEQ ID NO. 190) | (SEQ ID NO. 225) |
| | | | CGAGACCTACTTCT | CTACTCCTTTCTGGCCT | AGTCCCTCGCAATTTC |
| 14 | CCL5 | NM_002985 | TGCCCACATCAAG | GATGTACTCCCGAACC | AGCAGTCGTCTTTGTC |
| | | | GAGTATTT | CATTT | ACCCGAAA |
| | | | (SEQ ID NO. 191) | (SEQ ID NO. 192) | (SEQ ID NO. 226) |
| 15 | CD2 | NM_001767 | CCATCACACCAGT | GCATCTACACATGACC | AGAATGGTAGAGGAC |
| | | | AAGGAGAAG | TGAGAG | CGAGCACAGA |
| | | | (SEQ ID NO. 193) | (SEQ ID NO. 194) | (SEQ ID NO. 227) |
| 16 | CXCL13 | NM_006419 | CATCTCGACATCTC | GCTCTCTTGGACACAT | AGCCTCTCTCCAGTCC |
| | | | TGCTTCTC | CTACAC | AAGGTGTT |
| | | | (SEQ ID NO. 195) | (SEQ ID NO. 196) | (SEQ ID NO. 228) |
| 17 | GZMA | NM_006144 | GAGACTCGTGCAA | CGAGGGTCTCCGCATT | CCCTTTGTTGTGCGAG |
| | | | TGGAGATT | TATT | GGTGTTT |
| | | | (SEQ ID NO. 197) | (SEQ ID NO. 198) | (SEQ ID NO. 229) |
| 18 | MNDA | NM_002432 | CCCACCGCAAGAA | TGCTCTTGGGACACCT | ACATCGGAAGCAAGA |
| | | | ACAAAC | TATTC | GGGAGGATT |
| | | | (SEQ ID NO. 199) | (SEQ ID NO. 200) | (SEQ ID NO. 230) |
| 19 | CD79A | NM_001783 | CCCACTCTTCTTCC | CACTAACGAGGCTGCT | CCCAGCGGGTAATGA |
| | | | CTCTAAAC | ACAA | GCCCTTAAT |
| | | | (SEQ ID NO. 201) | (SEQ ID NO. 202) | (SEQ ID NO. 231) |
| 20 | IGKV1-17 | ENSE00002515620 | GAGACAGAGTCAC | CAGCATAGATCAGGCG | TATCAGCAGAAACCA |
| | | | CATCACTTG | CTTAG | GGGAAAGCCC |
| | | | (SEQ ID NO. 203) | (SEQ ID NO. 204) | (SEQ ID NO. 232) |
| 21 | IGKV2-28 | ENSE00002466064 | CAGAGCCTCCTGC | GCCCGATTAGAACCCA | TTGGATTGGTACCTGC |
| | | | ATAGTAATG | AATAGA | AGAAGCCA |
| | | | (SEQ ID NO. 205) | (SEQ ID NO. 206) | (SEQ ID NO. 233) |
| 22 | GAPDH | NM_002046 | GGTGTGAACCATG | GAGTCCTTCCACGATA | AGATCATCAGCAATG |
| | | | AGAAGTATGA | CCAAAG | CCTCCTGCA |
| | | | (SEQ ID NO. 207) | (SEQ ID NO. 208) | (SEQ ID NO. 234) |
| 23 | GUSB | NM_000181 | TGCTGGCTACTACT | CCTTGTCTGCTGCATA | TCGCTCACACCAAAT |
| | | | TGAAGATG | GTTAGA | CCTTGGACC |
| | | | (SEQ ID NO. 209) | (SEQ ID NO. 210) | (SEQ ID NO. 235) |
| 24 | TFRC | NM_003234 | TTTCCACCATCTCG | GGGACAGTCTCCTTCC | CAGACAATCTCCAGA |
| | | | GTCATC | ATATTC | GCTGCTGCA |
| | | | (SEQ ID NO. 211) | (SEQ ID NO. 212) | (SEQ ID NO. 236) |

### Example 5: Prediction of Chemotherapy Benefit in a Patient with Colon Cancer Based on the Results of Colorectal Cancer Molecular Subtyping and Risk Assessment

Procedures: Risk assessment of 281 stage III colon cancer cases was performed using the 24-gene testing combination for colorectal cancer molecular subtyping and risk assessment. Specifically, recurrence risk was assessed for each colon cancer case using the method described in Example 2; then the Kaplan-Meier method was used to compare the difference in survival curves between the groups with and without chemotherapy.

Results: the 281 cases of stage III colon cancer were subjected to recurrence risk assessment and classified into low risk group (108 cases) and high risk group (173 cases) (Table 5).

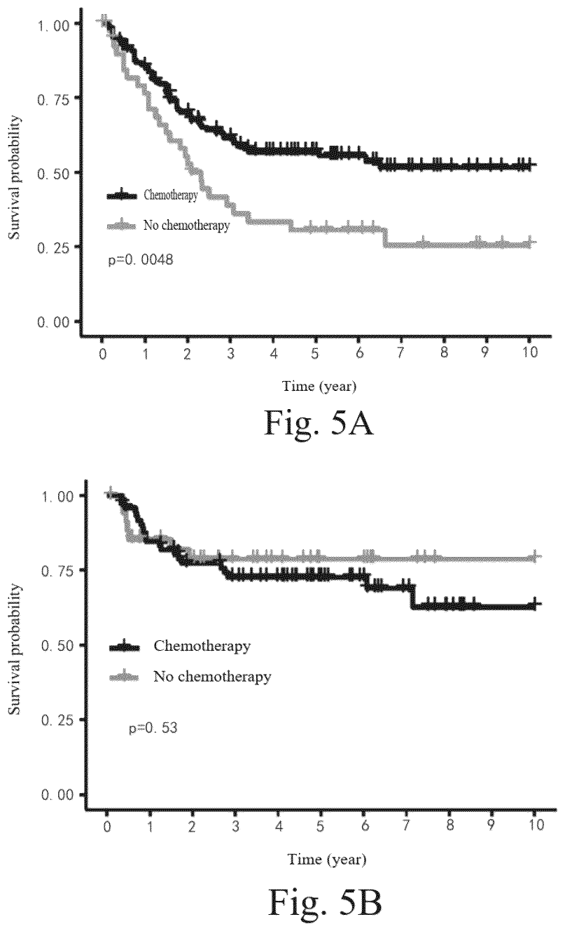

The results of survival analysis using the Kaplan-Meier method for the cases in the high risk group are shown in FIG. 5A, and those for the cases in the low risk group are shown in FIG. 5B. The results show that for the stage III colon cancer cases with high risk according to the recurrence risk assessment, the 10-year distant metastasis-free survival rate was higher in the group receiving chemotherapy than that in the group not receiving chemotherapy (FIG. 5A); while for the stage III colon cancer cases with low risk according to the recurrence risk assessment, there was no significant difference in the 10-year distant metastasis-free survival rates between the group receiving and the group not receiving chemotherapy (FIG. 5B). That is, stage III colon cancer patients assessed as high risk according to the method of the present disclosure can benefit from chemotherapy. Therefore, the gene panel according to the present disclosure can be used to determine the molecular subtype of colorectal cancer and/or assess the survival risk of a patient with colorectal cancer. Based on the results of survival risk assessment, it is possible to predict whether a patient with colorectal cancer will benefit from chemotherapy.

TABLE 5

| Risk Group | Number |
|---|---|
| Low risk | 108 |
| High risk | 173 |
| Total | 281 |

### Example 6: Distribution of Colorectal Cancer Genetic Mutations in Different Molecular Subtypes

Procedures: Molecular subtyping was performed on 364 colon cancer cases using the 24-gene testing combination for colorectal cancer molecular subtyping and risk assessment. Specifically, molecular subtyping was performed for each colorectal cancer case using the method described in Example 2; then the distribution of genetic mutations in each molecular subtype was subjected to statistical analysis based on the genetic mutation information in the TCGA database.

Results: the 364 colon cancer cases were subjected to molecular subtyping and categorized into CRC1, CRC2, CRC3, CRC4, CRC5 and Mixed subtypes, and BRAF, ERBB2, KDR, KRAS and VEGFA mutations were distributed differently in different subtypes (Table 6).

TABLE 6

| | subtype | | | | | |
|---|---|---|---|---|---|---|
| mutation/ | CRC1 | CRC2 | CRC3 | CRC4 | CRC5 | Mixed |
| MMR | (n = 68) | (n = 67) | (n = 97) | (n = 45) | (n = 56) | (n = 31) |
| BRAF | 7 (10%) | 14 (21%) | 10 (10%) | 22 (49%) | 0% | 5 (16%) |
| ERBB2 | 1 (1%) | 10 (15%) | 5 (5%) | 3 (7%) | 2 (4%) | 3 (10%) |
| KDR | 4 (6%) | 6 (9%) | 3 (3%) | 6 (13%) | 1 (2%) | 3 (10%) |
| KRAS | 25 (37%) | 27 (40%) | 43 (44%) | 17 (38%) | 25 (45%) | 9 (29%) |
| VECRCFA | 2 (3%) | 4 (6%) | 1 (1%) | 3 (7%) | 0% | 1 (3%) |

1-24. (canceled)

25. A method of determining the molecular subtype of colorectal cancer and/or the survival risk of a patient with colorectal cancer, wherein the method comprises the following steps:
(1) providing a test sample of the patient;
(2) determining the expression levels of the molecular subtype and survival risk assessment related genes in a gene panel in the test sample, wherein the molecular subtype and survival risk assessment related genes in the gene panel comprise proliferation-related genes, extracellular matrix-related genes, intracellular matrix-related genes, immune-related genes and immunoglobulin-related genes;
(3) calculating a gene expression profile for the test sample based on the expression levels determined in step (2), wherein the gene expression profile is calculated based on the expression levels of the proliferation-related genes, extracellular matrix-related genes, intracellular matrix-related genes and immune-related genes;
(4) calculating an immunoglobulin index for the test sample according to the expression levels of the immunoglobulin-related genes determined in step (2);
(5) determining a mismatch repair (MMR) index for the test sample according to the mismatch repair status of the test sample, wherein the MMR index is assigned to 1 when the MMR status is proficient mismatch repair (pMMR), and the MMR index is assigned to −1 when the MMR status is deficient mismatch repair (dMMR);
(6) obtaining the expression levels of the molecular subtype and survival risk assessment related genes used in step (2) for each of the samples in a training set of colorectal cancer samples, wherein the survival data of each of the samples in the training set are known;
(7) calculating a gene expression profile for each of the samples in the training set based on the expression levels obtained in step (6), wherein the gene expression profile is calculated based on the expression levels of the proliferation-related genes, extracellular matrix-related genes, intracellular matrix-related genes and immune-related genes;
(8) classifying each of the samples in the training set into CRC1 subtype, CRC2 subtype, CRC3 subtype, CRC4 subtype, CRC5 subtype or Mixed subtype by comparing the similarity of the gene expression profiles among the samples in the training set and calculating the gene expression profile for the CRC1 subtype, CRC2 subtype, CRC3 subtype, CRC4 subtype and CRC5 subtype colorectal cancer samples;
(9) calculating an immunoglobulin index for each of the samples in the training set according to the expression levels of the immunoglobulin-related genes obtained in step (6);
(10) determining a mismatch repair (MMR) index for each of the samples in the training set according to the mismatch repair status of each of the samples in the training set, wherein the MMR index is assigned to 1 when the MMR status is proficient mismatch repair (pMMR), and the MMR index is assigned to −1 when the MMR status is deficient mismatch repair (dMMR);
(11) calculating a survival risk score for each of the samples in the training set by fitting a model to the gene expression profiles for the CRC1 subtype, CRC2 subtype, CRC3 subtype, CRC4 subtype and CRC5 subtype colorectal cancer samples obtained in step (8), the gene expression profile in each of the samples in the training set obtained in step (7), the immunoglobulin index obtained in step (9), and the MMR index obtained in step (10);
(12) assigning the risk scores calculated in step (11) to high or low risk groups with an appropriate cutoff value based on the survival data of each of the samples in the training set;
(13) calculating a survival risk score for the test sample using the model of step (11); and
(14) determining which risk group the risk score calculated in step (13) falls within by comparing the risk score calculated in step (13) to the risk groups assigned in step (12), wherein assignment to a high risk group indicates the patient having a high risk of progression, and assignment to a low risk group indicates the patient having a low risk of progression;
wherein the gene panel comprises 21 molecular subtyping and survival risk assessing related genes of the following:
(i) the following proliferation-related genes: CCNB2, MKI67, RRM1, SPAG5 and TOP2A;
(ii) the following extracellular matrix-related genes: AEBP1, COL6A3, HTRA1, MMP2 and TIMP3;
(iii) the following intracellular matrix-related genes: ADNP, MAPRE1 and TMEM189-UBE2V1;
(iv) the following immune-related genes: CCL5, CD2, CXCL13, GZMA and MNDA; and
(v) the following immunoglobulin-related genes: CD79A, IGKV1-17 and IGKV2-28.

26. The method according to claim 25, wherein
the gene panel comprises 76 molecular subtyping and survival risk assessing related genes of the following:
(i) the following proliferation-related genes: CCNB2, MKI67, RRM1, SPAG5, TOP2A, CKS1B, DNMT1, DTYMK, EZH2, FOXM1, MAD2L1, MCM2, MCM3, MCM6, PCLAF, PLK1, PSRC1, RFC5, SMC4, TMPO and UBE2S;
(ii) the following extracellular matrix-related genes: AEBP1, COL6A3, HTRA1, MMP2, TIMP3, CLIC4, DPYSL3, EFEMP1, GJA1, LGALS1, LUM, MSN, PALLD, SERPING1, TIMP1, TNC and VIM;
(iii) the following intracellular matrix-related genes: ADNP, MAPRE1, TMEM189-UBE2V1, CSE1L, EIF2S2, EIF6, NCOA6, PPP1R3D, PRPF6, PSMA7, RALY, RBM39, RNF114, RPS21, TOMM34 and ZMYND8;
(iv) the following immune-related genes: CCL5, CD2, CXCL13, GZMA, MNDA, BCL2A1, CCL3, CSF2RB, LCP2, PLA2G7, RASGRP1, RHOH and TLR2; and
(v) the following immunoglobulin-related genes: CD79A, IGKV1-17, IGKV2-28, CD27, IGHM, IGKV4-1, JCHAIN, POU2AF1 and TNFRSF17.

27. The method according to claim 25, wherein
the gene panel further comprises a reference gene(s) and the expression levels of the molecular subtype and survival risk assessment related genes are normalized to the reference gene(s).

28. The method according to claim 27, wherein
the reference gene(s) comprises at least one of the following reference genes:
GAPDH, GUSB, TFRC, MRPL19, PSMC4 and SF3A1.

29. The method according to claim 25, wherein
the gene panel comprises:
(a) 21 molecular subtyping and survival risk assessing related genes of the following:
(i) the following proliferation-related genes: CCNB2, MKI67, RRM1, SPAG5 and TOP2A;
(ii) the following extracellular matrix-related genes: AEBP1, COL6A3, HTRA1, MMP2 and TIMP3;
(iii) the following intracellular matrix-related genes: ADNP, MAPRE1 and TMEM189-UBE2V1;
(iv) the following immune-related genes: CCL5, CD2, CXCL13, GZMA and MNDA; and
(v) the following immunoglobulin-related genes: CD79A, IGKV1-17 and IGKV2-28; and
three of the following reference genes: GAPDH, GUSB, TFRC, MRPL19, PSMC4 and SF3A1;
or
(b) 76 molecular subtyping and survival risk assessing related genes of the following:
(i) the following proliferation-related genes: CCNB2, MKI67, RRM1, SPAG5, TOP2A, CKS1B, DNMT1, DTYMK, EZH2, FOXM1, MAD2L1, MCM2, MCM3, MCM6, PCLAF, PLK1, PSRC1, RFC5, SMC4, TMPO and UBE2S;
(ii) the following extracellular matrix-related genes: AEBP1, COL6A3, HTRA1, MMP2, TIMP3, CLIC4, DPYSL3, EFEMP1, GJA1, LGALS1, LUM, MSN, PALLD, SERPING1, TIMP1, TNC and VIM;
(iii) the following intracellular matrix-related genes: ADNP, MAPRE1, TMEM189-UBE2V1, CSE1L, EIF2S2, EIF6, NCOA6, PPP1R3D, PRPF6, PSMA7, RALY, RBM39, RNF114, RPS21, TOMM34 and ZMYND8;
(iv) the following immune-related genes: CCL5, CD2, CXCL13, GZMA, MNDA, BCL2A1, CCL3, CSF2RB, LCP2, PLA2G7, RASGRP1, RHOH and TLR2; and
(v) the following immunoglobulin-related genes: CD79A, IGKV1-17, IGKV2-28, CD27, IGHM, IGKV4-1, JCHAIN, POU2AF1 and TNFRSF17; and
the following reference genes: GAPDH, GUSB, TFRC, MRPL19, PSMC4 and SF3A1.

30. A method of determining the molecular subtype of colorectal cancer in a subject, wherein the method comprises the following steps:
(1) providing a test sample of the subject;
(2) determining the expression levels of the molecular subtype and survival risk assessment related genes in a gene panel in the test sample, wherein the molecular subtype and survival risk assessment related genes in the gene panel comprise proliferation-related genes, extracellular matrix-related genes, intracellular matrix-related genes, and immune-related genes;
(3) calculating a gene expression profile for the test sample based on the expression levels determined in step (2), where the gene expression profile is calculated based on the expression levels of the proliferation-related genes, extracellular matrix-related genes, intracellular matrix-related genes and immune-related genes;
(4) obtaining the expression levels of the molecular subtype and survival risk assessment related genes used in step (2) for each of the samples in a training set of colorectal cancer samples, wherein the survival data of each of the samples in the training set are known;
(5) calculating a gene expression profile for each of the samples in the training set based on the expression levels obtained in step (4), wherein the gene expression profile is calculated based on the expression levels of the proliferation-related genes, extracellular matrix-related genes, intracellular matrix-related genes and immune-related genes;
(6) classifying each of the samples in the training set into CRC1 subtype, CRC2 subtype, CRC3 subtype, CRC4 subtype, CRC5 subtype or mixed subtype by comparing the similarity of the gene expression profiles among the samples in the training set and calculating the gene expression profile for the CRC1 subtype, CRC2 subtype, CRC3 subtype, CRC4 subtype and CRC5 subtype colorectal cancer samples;
(7) calculating the correlation coefficient between the gene expression profile in the test sample calculated in step (3) and the gene expression profile in the CRC1 subtype, CRC2 subtype, CRC3 subtype, CRC4 subtype, CRC5 subtype colorectal cancer samples obtained in step (6); and
(8) determining the test sample as X subtype when the correlation coefficient between the gene expression profile in the test sample and the gene expression profile in X subtype is the highest and the confidence limit is greater than or equal to 0.8, wherein X is selected from colorectal cancer; and determining the test sample as a mixed subtype when the confidence limit is lower than 0.8;
wherein the gene panel comprises molecular subtyping and survival risk assessing related genes of the following:
(i) the following proliferation-related genes: CCNB2, MKI67, RRM1, SPAG5 and TOP2A;
(ii) the following extracellular matrix-related genes: AEBP1, COL6A3, HTRA1, MMP2 and TIMP3;
(iii) the following intracellular matrix-related genes: ADNP, MAPRE1 and TMEM189-UBE2V1; and
(iv) the following immune-related genes: CCL5, CD2, CXCL13, GZMA and MNDA.

31. The method according to claim 30, wherein
the gene panel comprises molecular subtyping and survival risk assessing related genes of the following:
(i) the following proliferation-related genes: CCNB2, MKI67, RRM1, SPAG5, TOP2A, CKS1B, DNMT1, DTYMK, EZH2, FOXM1, MAD2L1, MCM2, MCM3, MCM6, PCLAF, PLK1, PSRC1, RFC5, SMC4, TMPO and UBE2S;
(ii) the following extracellular matrix-related genes: AEBP1, COL6A3, HTRA1, MMP2, TIMP3, CLIC4, DPYSL3, EFEMP1, GJA1, LGALS1, LUM, MSN, PALLD, SERPING1, TIMP1, TNC and VIM;
(iii) the following intracellular matrix-related genes: ADNP, MAPRE1, TMEM189-UBE2V1, CSE1L, EIF2S2, EIF6, NCOA6, PPP1R3D, PRPF6, PSMA7, RALY, RBM39, RNF114, RPS21, TOMM34 and ZMYND8; and
(iv) the following immune-related genes: CCL5, CD2, CXCL13, GZMA, MNDA, BCL2A1, CCL3, CSF2RB, LCP2, PLA2G7, RASGRP1, RHOH and TLR2.

32. The method according to claim 30, wherein
the gene panel further comprises a reference gene(s) and the expression levels of the molecular subtype and survival risk assessment related genes are normalized to the reference gene(s).

33. The method according to claim 32, wherein
the reference gene(s) comprises at least one of the following reference genes:
GAPDH, GUSB, TFRC, MRPL19, PSMC4 and SF3A1.

34. The method according to claim 30, wherein
the gene panel comprises:
(a) molecular subtyping and survival risk assessing related genes of the following:
(i) the following proliferation-related genes: CCNB2, MKI67, RRM1, SPAG5 and TOP2A;
(ii) the following extracellular matrix-related genes: AEBP1, COL6A3, HTRA1, MMP2 and TIMP3;
(iii) the following intracellular matrix-related genes: ADNP, MAPRE1 and TMEM189-UBE2V1; and
(iv) the following immune-related genes: CCL5, CD2, CXCL13, GZMA and MNDA; and
three of the following reference genes: GAPDH, GUSB, TFRC, MRPL19, PSMC4 and SF3A1;
or
(b) molecular subtyping and survival risk assessing related genes of the following:
(i) the following proliferation-related genes: CCNB2, MKI67, RRM1, SPAG5, TOP2A, CKS1B, DNMT1, DTYMK, EZH2, FOXM1, MAD2L1, MCM2, MCM3, MCM6, PCLAF, PLK1, PSRC1, RFC5, SMC4, TMPO and UBE2S;
(ii) the following extracellular matrix-related genes: AEBP1, COL6A3, HTRA1, MMP2, TIMP3, CLIC4, DPYSL3, EFEMP1, GJA1, LGALS1, LUM, MSN, PALLD, SERPING1, TIMP1, TNC and VIM;
(iii) the following intracellular matrix-related genes: ADNP, MAPRE1, TMEM189-UBE2V1, CSE1L, EIF2S2, EIF6, NCOA6, PPP1R3D, PRPF6, PSMA7, RALY, RBM39, RNF114, RPS21, TOMM34 and ZMYND8; and
(iv) the following immune-related genes: CCL5, CD2, CXCL13, GZMA, MNDA, BCL2A1, CCL3, CSF2RB, LCP2, PLA2G7, RASGRP1, RHOH and TLR2; and
the following reference genes: GAPDH, GUSB, TFRC, MRPL19, PSMC4 and SF3A1.

35. A method for treating colorectal cancer in a subject, wherein the method comprises the following steps:
determining the molecular subtype and/or survival risk of the subject using the method according to claim 25; and
treating the subject, wherein if the subject is in a high risk group, the subject is further treated with chemotherapy, radiotherapy or biologic therapy, preferably with chemotherapy, after the colorectal cancer surgery.

36. A method of determining the molecular subtype of colorectal cancer, comprising determining the expressions of a proliferation-related gene set, an extracellular matrix-related gene set, an immune-related gene set and an intercellular matrix-related gene set, wherein
(i) the proliferation-related gene set comprises: CCNB2, MKI67, RRM1, SPAG5 and TOP2A;
(ii) the extracellular matrix-related gene set comprises: AEBP1, COL6A3, HTRA1, MMP2 and TIMP3;
(iii) the intracellular matrix-related gene set comprises: ADNP, MAPRE1 and TMEM189-UBE2V1; and
(iv) the immune-related gene set comprises: CCL5, CD2, CXCL13, GZMA and MNDA.

37. The method according to claim 36, wherein
(i) the proliferation-related gene set comprises: CCNB2, MKI67, RRM1, SPAG5, TOP2A, CKS1B, DNMT1, DTYMK, EZH2, FOXM1, MAD2L1, MCM2, MCM3, MCM6, PCLAF, PLK1, PSRC1, RFC5, SMC4, TMPO and UBE2S;
(ii) the extracellular matrix-related gene set comprises: AEBP1, COL6A3, HTRA1, MMP2, TIMP3, CLIC4, DPYSL3, EFEMP1, GJA1, LGALS1, LUM, MSN, PALLD, SERPING1, TIMP1, TNC and VIM;
(iii) the intracellular matrix-related gene set comprises: ADNP, MAPRE1, TMEM189-UBE2V1, CSE1L, EIF2S2, EIF6, NCOA6, PPP1R3D, PRPF6, PSMA7, RALY, RBM39, RNF114, RPS21, TOMM34 and ZMYND8; and
(iv) the immune-related gene set comprises: CCL5, CD2, CXCL13, GZMA, MNDA, BCL2A1, CCL3, CSF2RB, LCP2, PLA2G7, RASGRP1, RHOH and TLR2.

38. The method according to claim 36, wherein
the expressions of the proliferation-related gene set, the extracellular matrix-related gene set, the intracellular matrix-related gene set and the immune-related gene set are normalized to a reference gene(s).

39. The method according to claim 36, wherein based on the gene expression, the colorectal cancer is classified as either CRC1 subtype, CRC2 subtype, CRC3 subtype, CRC4 subtype, CRC5 subtype or Mixed subtype, wherein
the CRC1 subtype is characterized in low expression of the proliferation-related genes, high expression of the extracellular matrix-related genes, low expression of the immune-related genes, low expression of the intracellular matrix-related genes;
the CRC2 subtype is characterized in medium expression of the proliferation-related genes, low expression of the extracellular matrix-related genes, high expression of the immune-related genes, low expression of the intracellular matrix-related genes;
the CRC3 subtype is characterized in high expression of the proliferation-related genes, low expression of the extracellular matrix-related genes, low expression of the immune-related genes, high expression of the intracellular matrix-related genes;
the CRC4 subtype is characterized in low expression of the proliferation-related genes, low expression of the extracellular matrix-related genes, high expression of the immune-related genes, low expression of the intracellular matrix-related genes;
the CRC5 subtype is characterized in medium expression of the proliferation-related genes, high expression of the extracellular matrix-related genes, low expression of the immune-related genes, medium expression of the intracellular matrix-related genes; and
the Mixed subtype is the colorectal cancer not belonging to the CRC1 subtype, the CRC2 subtype, the CRC3 subtype, the CRC4 subtype and the CRC5 subtype.

40. A method of treating a patient for colorectal cancer comprising determining the molecular subtype of the colorectal cancer using the method according to claim 36; and
treating the patient based on the molecular subtype.

41. The method according to claim 40, wherein the colorectal cancer is classified as CRC1 or CRC5 subtype, and the patient is treated with chemotherapy, radiotherapy or biological therapy, preferably with chemotherapy.

42. A method of treating a patient for colorectal cancer comprising
providing a tumor sample of the patient,
determining the expression levels of the genes in an immunoglobulin-related gene set in the tumor sample,
comparing the expression levels of the genes in the tumor sample as determined to the expression levels of the genes in a reference colorectal cancer sample, and
after colorectal cancer surgery, further treating the patient having decreased expression levels of the genes compared to the reference colorectal cancer sample with chemotherapy, radiotherapy or biological therapy, preferably with chemotherapy;
wherein the immunoglobulin-related gene set comprises the following genes: CD79A, IGKV1-17 and IGKV2-28.

43. The method according to claim 42, wherein the immunoglobulin-related gene set comprises the following genes: CD79A, IGKV1-17, IGKV2-28, CD27, IGHM, IGKV4-1, JCHAIN, POU2AF1 and TNFRSF17.


SEQUENCE LISTING (236 sequences)

SEQ ID NO: 1 - DNA, 29 bp, Artificial Sequence
  CCNB2 upstream primer sequence
aatgtggtgaaagtaaatgaaaacttaac

SEQ ID NO: 2 - DNA, 25 bp, Artificial Sequence
  CCNB2 dowstream primer sequence
caagcagcaaactcctgaagatcag

SEQ ID NO: 3 - DNA, 27 bp, Artificial Sequence
  CKS1B upstream primer sequence
gatgggtccattatatgatccatgaac

SEQ ID NO: 4 - DNA, 23 bp, Artificial Sequence
  CKS1B dowstream primer sequence
aacctcacatcttgctgttccgg

SEQ ID NO: 5 - DNA, 25 bp, Artificial Sequence
  DNMT1 upstream primer sequence
ttggccaaagcccgagagagtgcct

SEQ ID NO: 6 - DNA, 25 bp, Artificial Sequence
  DNMT1 dowstream primer sequence
aataaaggaggaggaagctgctaag

SEQ ID NO: 7 - DNA, 28 bp, Artificial Sequence
  DTYMK upstream primer sequence
accgcgccgaactgctccggttcccgga

SEQ ID NO: 8 - DNA, 25 bp, Artificial Sequence
  DTYMK dowstream primer sequence
atcaactgaaatcggcaaacttctg

SEQ ID NO: 9 - DNA, 25 bp, Artificial Sequence
  EZH2 upstream primer sequence
caagaggttcagacgagctgatgaa

SEQ ID NO: 10 - DNA, 30 bp, Artificial Sequence
  EZH2 dowstream primer sequence
agagtatgtttagttccaatcgtcagaaaa

SEQ ID NO: 11 - DNA, 23 bp, Artificial Sequence
  FOXM1 upstream primer sequence
cggagctacggcctaacggcggc

SEQ ID NO: 12 - DNA, 25 bp, Artificial Sequence
  FOXM1 dowstream primer sequence
caatggagagtgaaaacgcagattc

SEQ ID NO: 13 - DNA, 23 bp, Artificial Sequence
  MAD2L1 upstream primer sequence
gcgggagcgccgaaatcgtggcc

SEQ ID NO: 14 - DNA, 25 bp, Artificial Sequence
  MAD2L1 dowstream primer sequence
aacagcattttatatcagcgtggca

SEQ ID NO: 15 - DNA, 25 bp, Artificial Sequence
  MCM2 upstream primer sequence
tgaggtccctgagaaggacttggtg

SEQ ID NO: 16 - DNA, 29 bp, Artificial Sequence
  MCM2 dowstream primer sequence
atccacaacctctctgcattttatgacag

SEQ ID NO: 17 - DNA, 25 bp, Artificial Sequence
  MCM3 upstream primer sequence
gagattacctggacttcctggacga

SEQ ID NO: 18 - DNA, 29 bp, Artificial Sequence
  MCM3 dowstream primer sequence
ggaagaccagggaatttatcagagcaaag

SEQ ID NO: 19 - DNA, 25 bp, Artificial Sequence
  MCM6 upstream primer sequence
agaaactgttcctggacttcttgga

SEQ ID NO: 20 - DNA, 28 bp, Artificial Sequence
  MCM6 dowstream primer sequence
tttcagagcagcgatggagaaattaaat

SEQ ID NO: 21 - DNA, 29 bp, Artificial Sequence
  MKI67 upstream primer sequence
aacctctgctccccacctcagagagtttt

SEQ ID NO: 22 - DNA, 26 bp, Artificial Sequence
  MKI67 dowstream primer sequence
gaggaaatgtgttcttcagtgcacag

SEQ ID NO: 23 - DNA, 25 bp, Artificial Sequence
  PCLAF upstream primer sequence
taaagcagacagtgttccaggcact

SEQ ID NO: 24 - DNA, 25 bp, Artificial Sequence
  PCLAF dowstream primer sequence
tggtggctgctcgagcccccagaaa

SEQ ID NO: 25 - DNA, 25 bp, Artificial Sequence
  PLK1 upstream primer sequence
gcagcgtgcagatcaacttcttcca

SEQ ID NO: 26 - DNA, 25 bp, Artificial Sequence
  PLK1 dowstream primer sequence
tcacaccaagctcatcttgtgccca

SEQ ID NO: 27 - DNA, 25 bp, Artificial Sequence
  PSRC1 upstream primer sequence
tggctggacatggaggatttggagg

SEQ ID NO: 28 - DNA, 29 bp, Artificial Sequence
  PSRC1 dowstream primer sequence
atgtaaggtttattgtggatgagaccttg

SEQ ID NO: 29 - DNA, 28 bp, Artificial Sequence
  RFC5 upstream primer sequence
aatgatctcatttctcatcaggacattc

SEQ ID NO: 30 - DNA, 25 bp, Artificial Sequence
  RFC5 dowstream primer sequence
tcaatgaagaccgactgccacactt

SEQ ID NO: 31 - DNA, 21 bp, Artificial Sequence
  RRM1 upstream primer sequence
atgcacttctacggctggaag

SEQ ID NO: 32 - DNA, 29 bp, Artificial Sequence
  RRM1 dowstream primer sequence
ggtttgaagactgggatgtattatttaag

SEQ ID NO: 33 - DNA, 23 bp, Artificial Sequence
  SMC4 upstream primer sequence
cgcacggagagcccagccaccgc

SEQ ID NO: 34 - DNA, 28 bp, Artificial Sequence
  SMC4 dowstream primer sequence
agactgcaagtgaggaacttgataatag

SEQ ID NO: 35 - DNA, 29 bp, Artificial Sequence
  SPAG5 upstream primer sequence
agaaaaactagatgacattgttcagcata

SEQ ID NO: 36 - DNA, 25 bp, Artificial Sequence
  SPAG5 dowstream primer sequence
aggtggtgaggggatgcaaagaact

SEQ ID NO: 37 - DNA, 25 bp, Artificial Sequence
  TMPO upstream primer sequence
tgaaatacggagtgaatcctggtcc

SEQ ID NO: 38 - DNA, 31 bp, Artificial Sequence
  TMPO dowstream primer sequence
agctatatgagaaaaagcttttgaaactgag

SEQ ID NO: 39 - DNA, 27 bp, Artificial Sequence
  TOP2A upstream primer sequence
aagaagacttggctacatttattgaag

SEQ ID NO: 40 - DNA, 26 bp, Artificial Sequence
  TOP2A dowstream primer sequence
aaacaagatgaacaagtcggacttcc

SEQ ID NO: 41 - DNA, 25 bp, Artificial Sequence
  UBE2S upstream primer sequence
cctcaccgacctccaggtcaccatc

SEQ ID NO: 42 - DNA, 25 bp, Artificial Sequence
  UBE2S dowstream primer sequence
ggaccccatatgctggaggtctgtt

SEQ ID NO: 43 - DNA, 25 bp, Artificial Sequence
  AEBP1 upstream primer sequence
ggcaagccagggaagcggccaggga

SEQ ID NO: 44 - DNA, 25 bp, Artificial Sequence
  AEBP1 dowstream primer sequence
gcctccggaaaagaccaaagacaaa

SEQ ID NO: 45 - DNA, 25 bp, Artificial Sequence
  CLIC4 upstream primer sequence
aagagcccctcatcgagctcttcgt

SEQ ID NO: 46 - DNA, 25 bp, Artificial Sequence
  CLIC4 dowstream primer sequence
gcagtgatggtgaaagcataggaaa

SEQ ID NO: 47 - DNA, 25 bp, Artificial Sequence
  COL6A3 upstream primer sequence
agcccagggacacacgccttcaggt

SEQ ID NO: 48 - DNA, 29 bp, Artificial Sequence
  COL6A3 dowstream primer sequence
tttggtgcttagacaaattcaaaatgagg

SEQ ID NO: 49 - DNA, 30 bp, Artificial Sequence
  DPYSL3 upstream primer sequence
tttatgctgatatttacatggaagatggct

SEQ ID NO: 50 - DNA, 25 bp, Artificial Sequence
  DPYSL3 dowstream primer sequence
ttggagacaatctgattgttcctgg

SEQ ID NO: 51 - DNA, 25 bp, Artificial Sequence
  EFEMP1 upstream primer sequence
aggacaccgaagaaaccatcacgta

SEQ ID NO: 52 - DNA, 25 bp, Artificial Sequence
  EFEMP1 dowstream primer sequence
atgcactgacggatatgagtgggat

SEQ ID NO: 53 - DNA, 25 bp, Artificial Sequence
  GJA1 upstream primer sequence
cacttggcgtgacttcactactttt

SEQ ID NO: 54 - DNA, 25 bp, Artificial Sequence
  GJA1 dowstream primer sequence
tggtgcccaggcaacatgggtgact

SEQ ID NO: 55 - DNA, 23 bp, Artificial Sequence
  HTRA1 upstream primer sequence
gtcctgcagcgcggagcctgcgg

SEQ ID NO: 56 - DNA, 25 bp, Artificial Sequence
  HTRA1 dowstream primer sequence
ggaagatcccaacagtttgcgccat

SEQ ID NO: 57 - DNA, 25 bp, Artificial Sequence
  LGALS1 upstream primer sequence
cgaggcgaggtggctcctgacgcta

SEQ ID NO: 58 - DNA, 25 bp, Artificial Sequence
  LGALS1 dowstream primer sequence
gcttcgtgctgaacctgggcaaaga

SEQ ID NO: 59 - DNA, 29 bp, Artificial Sequence
  LUM upstream primer sequence
aagaattaacgaaagcagtgtcaagacag

SEQ ID NO: 60 - DNA, 25 bp, Artificial Sequence
  LUM dowstream primer sequence
tttgccaaaaatgagtctaagtgca

SEQ ID NO: 61 - DNA, 25 bp, Artificial Sequence
  MMP2 upstream primer sequence
ctggatgccgtcgtggacctgcagg

SEQ ID NO: 62 - DNA, 25 bp, Artificial Sequence
  MMP2 dowstream primer sequence
tcaagggtgcctattacctgaagct

SEQ ID NO: 63 - DNA, 25 bp, Artificial Sequence
  MSN upstream primer sequence
actccgctgcctttgccgccaccat

SEQ ID NO: 64 - DNA, 25 bp, Artificial Sequence
  MSN dowstream primer sequence
cagtgtgcgtgtgaccaccatggat

SEQ ID NO: 65 - DNA, 25 bp, Artificial Sequence
  PALLD upstream primer sequence
caaggaggacctcctgaacaatggc

SEQ ID NO: 66 - DNA, 25 bp, Artificial Sequence
  PALLD dowstream primer sequence
agaaagaatggctcgtcgactgcta

SEQ ID NO: 67 - DNA, 25 bp, Artificial Sequence
  SERPING1 upstream primer sequence
ctgaccctgctgaccctcctgctgc

SEQ ID NO: 68 - DNA, 25 bp, Artificial Sequence
  SERPING1 dowstream primer sequence
gagcctcctcaaatccaaatgctac

SEQ ID NO: 69 - DNA, 25 bp, Artificial Sequence
  TIMP1 upstream primer sequence
cgcagatccagcgcccagagagaca

SEQ ID NO: 70 - DNA, 25 bp, Artificial Sequence
  TIMP1 dowstream primer sequence
cctttgagcccctggcttctggcat

SEQ ID NO: 71 - DNA, 28 bp, Artificial Sequence
  TIMP3 upstream primer sequence
ccacccccaggacgccttctgcaactcc

SEQ ID NO: 72 - DNA, 25 bp, Artificial Sequence
  TIMP3 dowstream primer sequence
tccgggccaaggtggtggggaagaa

SEQ ID NO: 73 - DNA, 25 bp, Artificial Sequence
  TNC upstream primer sequence
cttccaaggacctaggtctctcgcc

SEQ ID NO: 74 - DNA, 29 bp, Artificial Sequence
  TNC dowstream primer sequence
aaataattctttcaagaagatcagggaca

SEQ ID NO: 75 - DNA, 29 bp, Artificial Sequence
  VIM upstream primer sequence
acttctgattaagacggttgaaactagag

SEQ ID NO: 76 - DNA, 25 bp, Artificial Sequence
  VIM dowstream primer sequence
atcaacgaaacttctcagcatcacg

SEQ ID NO: 77 - DNA, 25 bp, Artificial Sequence
  ADNP upstream primer sequence
actgtgggacccatcacttacgaaa

SEQ ID NO: 78 - DNA, 25 bp, Artificial Sequence
  ADNP dowstream primer sequence
ttctgctgcagcgcttgtccatttt

SEQ ID NO: 79 - DNA, 25 bp, Artificial Sequence
  CSE1L upstream primer sequence
gctggggttccctcctccgtttctg

SEQ ID NO: 80 - DNA, 25 bp, Artificial Sequence
  CSE1L dowstream primer sequence
tcagcgatgcaaatctgcaaacact

SEQ ID NO: 81 - DNA, 25 bp, Artificial Sequence
  EIF2S2 upstream primer sequence
cactcgagccgcagccatgtctggg

SEQ ID NO: 82 - DNA, 28 bp, Artificial Sequence
  EIF2S2 dowstream primer sequence
gatttttgatcctactatgagcaagaag

SEQ ID NO: 83 - DNA, 25 bp, Artificial Sequence
  EIF6 upstream primer sequence
catgcgggattccctcattgacagc

SEQ ID NO: 84 - DNA, 25 bp, Artificial Sequence
  EIF6 dowstream primer sequence
acctgagtcaccttccaagttgttc

SEQ ID NO: 85 - DNA, 25 bp, Artificial Sequence
  MAPRE1 upstream primer sequence
ggcagtggacgcggttctgccgaga

SEQ ID NO: 86 - DNA, 25 bp, Artificial Sequence
  MAPRE1 dowstream primer sequence
cgtatactcaacgtcagtgaccagt

SEQ ID NO: 87 - DNA, 33 bp, Artificial Sequence
  NCOA6 upstream primer sequence
agaagatgacctggataaatgataaaaattaag

SEQ ID NO: 88 - DNA, 25 bp, Artificial Sequence
  NCOA6 dowstream primer sequence
tgtcctcttggcatatgcttctgga

SEQ ID NO: 89 - DNA, 25 bp, Artificial Sequence
  PPP1R3D upstream primer sequence
accgcaagaagcggaggacctggac

SEQ ID NO: 90 - DNA, 25 bp, Artificial Sequence
  PPP1R3D dowstream primer sequence
ggagcaaggtggcgaaccaagggta

SEQ ID NO: 91 - DNA, 25 bp, Artificial Sequence
  PRPF6 upstream primer sequence
acgacgaggatctaaatgacaccaa

SEQ ID NO: 92 - DNA, 25 bp, Artificial Sequence
  PRPF6 dowstream primer sequence
ctatgctgggagcctcttctcaagt

SEQ ID NO: 93 - DNA, 25 bp, Artificial Sequence
  PSMA7 upstream primer sequence
tgctgtcatgaggcgagatcaatcc

SEQ ID NO: 94 - DNA, 29 bp, Artificial Sequence
  PSMA7 dowstream primer sequence
agaaattgagaagtatgttgctgaaattg

SEQ ID NO: 95 - DNA, 25 bp, Artificial Sequence
  RALY upstream primer sequence
cgcggcttcctccagacctctcggc

SEQ ID NO: 96 - DNA, 25 bp, Artificial Sequence
  RALY dowstream primer sequence
gaggcaggtggtgctgaccctgtaa

SEQ ID NO: 97 - DNA, 25 bp, Artificial Sequence
  RBM39 upstream primer sequence
gagcaccacaggcgcccgaaggccg

SEQ ID NO: 98 - DNA, 29 bp, Artificial Sequence
  RBM39 dowstream primer sequence
agagaaaatggcagacgatattgatattg

SEQ ID NO: 99 - DNA, 25 bp, Artificial Sequence
  RNF114 upstream primer sequence
gcacagagacttcttgccatggctg

SEQ ID NO: 100 - DNA, 25 bp, Artificial Sequence
  RNF114 dowstream primer sequence
aagatccggtcccacgtggctactt

SEQ ID NO: 101 - DNA, 25 bp, Artificial Sequence
  RPS21 upstream primer sequence
tctctcgcgcgcggtgtggtggcag

SEQ ID NO: 102 - DNA, 25 bp, Artificial Sequence
  RPS21 dowstream primer sequence
agcccagcctcgaaatgcagaacga

SEQ ID NO: 103 - DNA, 25 bp, Artificial Sequence
  TMEM189-UBE2V1 upstream primer sequence
ccccacgagacctacttctgcatca

SEQ ID NO: 104 - DNA, 25 bp, Artificial Sequence
  TMEM189-UBE2V1 dowstream primer sequence
caatttccgactgttggaagaactc

SEQ ID NO: 105 - DNA, 25 bp, Artificial Sequence
  TOMM34 upstream primer sequence
taatgtgacgtcagccgtagaaggc

SEQ ID NO: 106 - DNA, 25 bp, Artificial Sequence
  TOMM34 dowstream primer sequence
tcatggactcgcttgggcctgagtg

SEQ ID NO: 107 - DNA, 29 bp, Artificial Sequence
  ZMYND8 upstream primer sequence
tgacattacacagtgttaacaatgcatcc

SEQ ID NO: 108 - DNA, 29 bp, Artificial Sequence
  ZMYND8 dowstream primer sequence
cttggctgaagaggaaataaaaacagaac

SEQ ID NO: 109 - DNA, 25 bp, Artificial Sequence
  BCL2A1 upstream primer sequence
ttgccccggatgtggatacctataa

SEQ ID NO: 110 - DNA, 28 bp, Artificial Sequence
  BCL2A1 dowstream primer sequence
tttcatattttgttgcggagttcataat

SEQ ID NO: 111 - DNA, 25 bp, Artificial Sequence
  CCL3 upstream primer sequence
gctctctgcaaccagttctctgcat

SEQ ID NO: 112 - DNA, 25 bp, Artificial Sequence
  CCL3 dowstream primer sequence
cttgctgctgacacgccgaccgcct

SEQ ID NO: 113 - DNA, 27 bp, Artificial Sequence
  CCL5 upstream primer sequence
gctactgccctctgcgctcctgcatct

SEQ ID NO: 114 - DNA, 26 bp, Artificial Sequence
  CCL5 dowstream primer sequence
cctcggacaccacaccctgctgcttt

SEQ ID NO: 115 - DNA, 26 bp, Artificial Sequence
  CD2 upstream primer sequence
caaggaatccagtgtcgagcctgtca

SEQ ID NO: 116 - DNA, 23 bp, Artificial Sequence
  CD2 dowstream primer sequence
tcatcattggcatatgtggagga

SEQ ID NO: 117 - DNA, 25 bp, Artificial Sequence
  CSF2RB upstream primer sequence
agaagactggtctctcccaccacac

SEQ ID NO: 118 - DNA, 25 bp, Artificial Sequence
  CSF2RB dowstream primer sequence
aggccaggagggagaggtcccaaga

SEQ ID NO: 119 - DNA, 25 bp, Artificial Sequence
  CXCL13 upstream primer sequence
ctgctggtcagcagcctctctccag

SEQ ID NO: 120 - DNA, 25 bp, Artificial Sequence
  CXCL13 dowstream primer sequence
tggaggtctattacacaagcttgag

SEQ ID NO: 121 - DNA, 24 bp, Artificial Sequence
  GZMA upstream primer sequence
aaagactgggtgttgactgcagct

SEQ ID NO: 122 - DNA, 26 bp, Artificial Sequence
  GZMA dowstream primer sequence
aacaaaaggtcccaggtcattcttgg

SEQ ID NO: 123 - DNA, 25 bp, Artificial Sequence
  LCP2 upstream primer sequence
ctgggaccccgacagccttgctgac

SEQ ID NO: 124 - DNA, 25 bp, Artificial Sequence
  LCP2 dowstream primer sequence
ctgtgagaaggcagtgaagaagtac

SEQ ID NO: 125 - DNA, 28 bp, Artificial Sequence
  MNDA upstream primer sequence
ctgaagactattgtggaagaagcatcca

SEQ ID NO: 126 - DNA, 29 bp, Artificial Sequence
  MNDA dowstream primer sequence
aagctataacatcagaaatggtgaatgaa

SEQ ID NO: 127 - DNA, 30 bp, Artificial Sequence
  PLA2G7 upstream primer sequence
aagcttcattagcattcttacaaaagcatt

SEQ ID NO: 128 - DNA, 25 bp, Artificial Sequence
  PLA2G7 dowstream primer sequence
ataaagattttgatcagtgggactg

SEQ ID NO: 129 - DNA, 25 bp, Artificial Sequence
  RASGRP1 upstream primer sequence
ctggacgatctcattgacagctgca

SEQ ID NO: 130 - DNA, 25 bp, Artificial Sequence
  RASGRP1 dowstream primer sequence
cctgtgtcgaagtaaccaactgttg

SEQ ID NO: 131 - DNA, 25 bp, Artificial Sequence
  RHOH upstream primer sequence
gaagccggctacaggaaattgactt

SEQ ID NO: 132 - DNA, 29 bp, Artificial Sequence
  RHOH dowstream primer sequence
aacttgctaatctcttttgtcacattcgg

SEQ ID NO: 133 - DNA, 25 bp, Artificial Sequence
  TLR2 upstream primer sequence
attgctctttcactgctttcaactg

SEQ ID NO: 134 - DNA, 25 bp, Artificial Sequence
  TLR2 dowstream primer sequence
tgaagcactggacaatgccacatac

SEQ ID NO: 135 - DNA, 25 bp, Artificial Sequence
  CD27 upstream primer sequence
aaagctgtgctgccagatgtgtgag

SEQ ID NO: 136 - DNA, 25 bp, Artificial Sequence
  CD27 dowstream primer sequence
gaaggactgtgaccagcatagaaag

SEQ ID NO: 137 - DNA, 25 bp, Artificial Sequence
  CD79A upstream primer sequence
cctcttcctgctgtctgctgtctac

SEQ ID NO: 138 - DNA, 25 bp, Artificial Sequence
  CD79A dowstream primer sequence
ggtgccaggccctgtggatgcacaa

SEQ ID NO: 139 - DNA, 23 bp, Artificial Sequence
  IGHM upstream primer sequence
gggtcaccgagaggaccgtggac

SEQ ID NO: 140 - DNA, 25 bp, Artificial Sequence
  IGHM dowstream primer sequence
agggggaggtgagcgccgacgagga

SEQ ID NO: 141 - DNA, 25 bp, Artificial Sequence
  IGKV1-17 upstream primer sequence
gagacagagtcaccatcacttgccg

SEQ ID NO: 142 - DNA, 25 bp, Artificial Sequence
  IGKV1-17 dowstream primer sequence
gcccctaagcgcctgatctatgctg

SEQ ID NO: 143 - DNA, 25 bp, Artificial Sequence
  IGKV2-28 upstream primer sequence
cagagcctcctgcatagtaatggat

SEQ ID NO: 144 - DNA, 25 bp, Artificial Sequence
  IGKV2-28 dowstream primer sequence
tgatctatttgggttctaatcgggc

SEQ ID NO: 145 - DNA, 25 bp, Artificial Sequence
  IGKV4-1 upstream primer sequence
ctccacagctcctgatctatttggg

SEQ ID NO: 146 - DNA, 25 bp, Artificial Sequence
  IGKV4-1 dowstream primer sequence
cactgaaaatcagcagagtggaggc

SEQ ID NO: 147 - DNA, 25 bp, Artificial Sequence
  JCHAIN upstream primer sequence
cctggcggtttttattaaggctgtt

SEQ ID NO: 148 - DNA, 29 bp, Artificial Sequence
  JCHAIN dowstream primer sequence
aagaagatgaaaggattgttcttgttgac

SEQ ID NO: 149 - DNA, 25 bp, Artificial Sequence
  POU2AF1 upstream primer sequence
tcctgtcacaggccatgctctggca

SEQ ID NO: 150 - DNA, 25 bp, Artificial Sequence
  POU2AF1 dowstream primer sequence
acccacagctccggagcaagcccca

SEQ ID NO: 151 - DNA, 25 bp, Artificial Sequence
  TNFRSF17 upstream primer sequence
aattaaccatttcgactcgagcagt

SEQ ID NO: 152 - DNA, 29 bp, Artificial Sequence
  TNFRSF17 dowstream primer sequence
atcttttgtcagaatagatgatgtgtcag

SEQ ID NO: 153 - DNA, 24 bp, Artificial Sequence
  GAPDH upstream primer sequence
tcaacgaccactttgtcaagctca

SEQ ID NO: 154 - DNA, 23 bp, Artificial Sequence
  GAPDH dowstream primer sequence
cagcaacagggtggtggacctca

SEQ ID NO: 155 - DNA, 22 bp, Artificial Sequence
  GUSB upstream primer sequence
gaggagcagtggtaccggcggc

SEQ ID NO: 156 - DNA, 27 bp, Artificial Sequence
  GUSB dowstream primer sequence
gacatgccagttccctccagcttcaat

SEQ ID NO: 157 - DNA, 25 bp, Artificial Sequence
  MRPL19 upstream primer sequence
ctgttcttccccttcgaggaatgaa

SEQ ID NO: 158 - DNA, 25 bp, Artificial Sequence
  MRPL19 dowstream primer sequence
tccacggggcggtgcttgtccacga

SEQ ID NO: 159 - DNA, 25 bp, Artificial Sequence
  PSMC4 upstream primer sequence
tctggggccgggacacggacagtgc

SEQ ID NO: 160 - DNA, 25 bp, Artificial Sequence
  PSMC4 dowstream primer sequence
cttctccaccaagatgcctatctcc

SEQ ID NO: 161 - DNA, 25 bp, Artificial Sequence
  SF3A1 upstream primer sequence
gaatcctcctttgaagatgcttctt

SEQ ID NO: 162 - DNA, 25 bp, Artificial Sequence
  SF3A1 dowstream primer sequence
ggctgtttgggctccgtgggcacgg

SEQ ID NO: 163 - DNA, 27 bp, Artificial Sequence
  TFRC upstream primer sequence
gtcatgaagaaactcaatgatcgtgtc

SEQ ID NO: 164 - DNA, 27 bp, Artificial Sequence
  TFRC dowstream primer sequence
tcctctctccctacgtatctccaaaag

SEQ ID NO: 165 - DNA, 20 bp, Artificial Sequence
  CCNB2 forward primer sequence
aaccagagcagcacaagtag

SEQ ID NO: 166 - DNA, 21 bp, Artificial Sequence
  CCNB2 reverse primer sequence
ggtttgacagaagcagtaggt

SEQ ID NO: 167 - DNA, 22 bp, Artificial Sequence
  MKI67 forward primer sequence
gacctcaaactggctcctaatc

SEQ ID NO: 168 - DNA, 22 bp, Artificial Sequence
  MKI67 reverse primer sequence
gctgccagatagagtcagaaag

SEQ ID NO: 169 - DNA, 20 bp, Artificial Sequence
  RRM1 forward primer sequence
tccacattgctgagcctaac

SEQ ID NO: 170 - DNA, 22 bp, Artificial Sequence
  RRM1 reverse primer sequence
ccgctggtcttgtccttaaata

SEQ ID NO: 171 - DNA, 20 bp, Artificial Sequence
  SPAG5 forward primer sequence
gccagcaccatagcagataa

SEQ ID NO: 172 - DNA, 21 bp, Artificial Sequence
  SPAG5 reverse primer sequence
agagagtcaggctctgtagtt

SEQ ID NO: 173 - DNA, 22 bp, Artificial Sequence
  TOP2A forward primer sequence
gacgcttcgttatgggaagata

SEQ ID NO: 174 - DNA, 20 bp, Artificial Sequence
  TOP2A reverse primer sequence
gggccagttgtgatggataa

SEQ ID NO: 175 - DNA, 20 bp, Artificial Sequence
  AEBP1 forward primer sequence
caccaacggctatgaggaaa

SEQ ID NO: 176 - DNA, 20 bp, Artificial Sequence
  AEBP1 reverse primer sequence
attccaggtgagtgggtaga

SEQ ID NO: 177 - DNA, 20 bp, Artificial Sequence
  COL6A3 forward primer sequence
caggtgaacctgggctaaat

SEQ ID NO: 178 - DNA, 22 bp, Artificial Sequence
  COL6A3 reverse primer sequence
gtctcccttctgtccaactatc

SEQ ID NO: 179 - DNA, 21 bp, Artificial Sequence
  HTRA1 forward primer sequence
gctagtgggtctgggtttatt

SEQ ID NO: 180 - DNA, 20 bp, Artificial Sequence
  HTRA1 reverse primer sequence
taagtggcaccgttcttcag

SEQ ID NO: 181 - DNA, 22 bp, Artificial Sequence
  MMP2 forward primer sequence
agagaacctcagggagagtaag

SEQ ID NO: 182 - DNA, 21 bp, Artificial Sequence
  MMP2 reverse primer sequence
cctcgaacagatgccacaata

SEQ ID NO: 183 - DNA, 21 bp, Artificial Sequence
  TIMP3 forward primer sequence
tttgcccttctcctccaatac

SEQ ID NO: 184 - DNA, 24 bp, Artificial Sequence
  TIMP3 reverse primer sequence
tctttcacacaccttgagtctatc

SEQ ID NO: 185 - DNA, 22 bp, Artificial Sequence
  ADNP forward primer sequence
gtctgctaatgcctcttctctc

SEQ ID NO: 186 - DNA, 22 bp, Artificial Sequence
  ADNP reverse primer sequence
tttggaactggactgacctaac

SEQ ID NO: 187 - DNA, 22 bp, Artificial Sequence
  MAPRE1 forward primer sequence
ggctgcgtattgtcagtttatg

SEQ ID NO: 188 - DNA, 22 bp, Artificial Sequence
  MAPRE1 reverse primer sequence
gttctggatgtactcgtgttct

SEQ ID NO: 189 - DNA, 21 bp, Artificial Sequence
  TMEM189-UBE2V1 forward primer sequence
cgagacctacttctgcatcac

SEQ ID NO: 190 - DNA, 22 bp, Artificial Sequence
  TMEM189-UBE2V1 reverse primer sequence
ctactcctttctggccttcttc

SEQ ID NO: 191 - DNA, 21 bp, Artificial Sequence
  CCL5 forward primer sequence
tgcccacatcaaggagtattt

SEQ ID NO: 192 - DNA, 21 bp, Artificial Sequence
  CCL5 reverse primer sequence
gatgtactcccgaacccattt

SEQ ID NO: 193 - DNA, 22 bp, Artificial Sequence
  CD2 forward primer sequence
ccatcacaccagtaaggagaag

SEQ ID NO: 194 - DNA, 22 bp, Artificial Sequence
  CD2 reverse primer sequence
gcatctacacatgacctgagag

SEQ ID NO: 195 - DNA, 22 bp, Artificial Sequence
  CXCL13 forward primer sequence
catctcgacatctctgcttctc

SEQ ID NO: 196 - DNA, 22 bp, Artificial Sequence
  CXCL13 reverse primer sequence
gctctcttggacacatctacac

SEQ ID NO: 197 - DNA, 21 bp, Artificial Sequence
  GZMA forward primer sequence
gagactcgtgcaatggagatt

SEQ ID NO: 198 - DNA, 20 bp, Artificial Sequence
  GZMA reverse primer sequence
cgagggtctccgcatttatt

SEQ ID NO: 199 - DNA, 19 bp, Artificial Sequence
  MNDA forward primer sequence
cccaccgcaagaaacaaac

SEQ ID NO: 200 - DNA, 21 bp, Artificial Sequence
  MNDA reverse primer sequence
tgctcttgggacaccttattc

SEQ ID NO: 201 - DNA, 22 bp, Artificial Sequence
  CD79A forward primer sequence
cccactcttcttccctctaaac

SEQ ID NO: 202 - DNA, 20 bp, Artificial Sequence
  CD79A reverse primer sequence
cactaacgaggctgctacaa

SEQ ID NO: 203 - DNA, 22 bp, Artificial Sequence
  IGKV1-17 forward primer sequence
gagacagagtcaccatcacttg

SEQ ID NO: 204 - DNA, 21 bp, Artificial Sequence
  IGKV1-17 reverse primer sequence
cagcatagatcaggcgcttag

SEQ ID NO: 205 - DNA, 22 bp, Artificial Sequence
  IGKV2-28 forward primer sequence
cagagcctcctgcatagtaatg

SEQ ID NO: 206 - DNA, 22 bp, Artificial Sequence
  IGKV2-28 reverse primer sequence
gcccgattagaacccaaataga

SEQ ID NO: 207 - DNA, 23 bp, Artificial Sequence
  GAPDH forward primer sequence
ggtgtgaaccatgagaagtatga

SEQ ID NO: 208 - DNA, 22 bp, Artificial Sequence
  GAPDH reverse primer sequence
gagtccttccacgataccaaag

SEQ ID NO: 209 - DNA, 22 bp, Artificial Sequence
  GUSB forward primer sequence
tgctggctactacttgaagatg

SEQ ID NO: 210 - DNA, 22 bp, Artificial Sequence
  GUSB reverse primer sequence
ccttgtctgctgcatagttaga

SEQ ID NO: 211 - DNA, 20 bp, Artificial Sequence
  TFRC forward primer sequence
tttccaccatctcggtcatc

SEQ ID NO: 212 - DNA, 22 bp, Artificial Sequence
  TFRC reverse primer sequence
gggacagtctccttccatattc

SEQ ID NO: 213 - DNA, 25 bp, Artificial Sequence
  CCNB2 probe sequence
accaaagttccagttcaacccacca

SEQ ID NO: 214 - DNA, 24 bp, Artificial Sequence
  MKI67 probe sequence
cgggagcagagccagtaaacttcc

SEQ ID NO: 215 - DNA, 24 bp, Artificial Sequence
  RRM1 probe sequence
agcagggtttgaagactgggatgt

SEQ ID NO: 216 - DNA, 23 bp, Artificial Sequence
  SPAG5 probe sequence
aaagctagggctgctgactgagc

SEQ ID NO: 217 - DNA, 24 bp, Artificial Sequence
  TOP2A probe sequence
atggttcccacatcaaaggcttgc

SEQ ID NO: 218 - DNA, 24 bp, Artificial Sequence
  AEBP1 probe sequence
ttcatgggaacgtggacaaggaca

SEQ ID NO: 219 - DNA, 24 bp, Artificial Sequence
  COL6A3 probe sequence
acaacaggacccaaaggcatcaga

SEQ ID NO: 220 - DNA, 24 bp, Artificial Sequence
  HTRA1 probe sequence
tggactgatcgtgacaaatgccca

SEQ ID NO: 221 - DNA, 27 bp, Artificial Sequence
  MMP2 probe sequence
tctgtcctgtagaaagagccctgaaga

SEQ ID NO: 222 - DNA, 24 bp, Artificial Sequence
  TIMP3 probe sequence
aggatcagtcaaaggcagcaagca

SEQ ID NO: 223 - DNA, 25 bp, Artificial Sequence
  ADNP probe sequence
tctctcagtcacaggcatccagagt

SEQ ID NO: 224 - DNA, 24 bp, Artificial Sequence
  MAPRE1 probe sequence
aatggagccagggaacagcatgtc

SEQ ID NO: 225 - DNA, 24 bp, Artificial Sequence
  TMEM189-UBE2V1 probe sequence
agtccctcgcaatttccgactgtt

SEQ ID NO: 226 - DNA, 24 bp, Artificial Sequence
  CCL5 probe sequence
agcagtcgtctttgtcacccgaaa

SEQ ID NO: 227 - DNA, 25 bp, Artificial Sequence
  CD2 probe sequence
agaatggtagaggaccgagcacaga

SEQ ID NO: 228 - DNA, 24 bp, Artificial Sequence
  CXCL13 probe sequence
agcctctctccagtccaaggtgtt

SEQ ID NO: 229 - DNA, 23 bp, Artificial Sequence
  GZMA probe sequence
ccctttgttgtgcgagggtgttt

SEQ ID NO: 230 - DNA, 24 bp, Artificial Sequence
  MNDA probe sequence
acatcggaagcaagagggaggatt

SEQ ID NO: 231 - DNA, 24 bp, Artificial Sequence
  CD79A probe sequence
cccagcgggtaatgagcccttaat

SEQ ID NO: 232 - DNA, 25 bp, Artificial Sequence
  IGKV1-17 probe sequence
tatcagcagaaaccagggaaagccc

SEQ ID NO: 233 - DNA, 24 bp, Artificial Sequence
  IGKV2-28 probe sequence
ttggattggtacctgcagaagcca

SEQ ID NO: 234 - DNA, 24 bp, Artificial Sequence
  GAPDH probe sequence
agatcatcagcaatgcctcctgca

SEQ ID NO: 235 - DNA, 24 bp, Artificial Sequence
  GUSB probe sequence
tcgctcacaccaaatccttggacc

SEQ ID NO: 236 - DNA, 24 bp, Artificial Sequence
  TFRC probe sequence
cagacaatctccagagctgctgca